(19) United States
(12) Patent Application Publication
(10) Pub. No.: US 2016/0228434 A1
(43) Pub. Date: Aug. 11, 2016

(54) TREATMENT OF DISEASES CAUSED BY ABNORMAL LYMPHOCYTE FUNCTION WITH AN HDAC6 INHIBITOR

(71) Applicants: ACETYLON PHARMACEUTICALS, INC., Boston, MA (US); EDWARD VIA COLLEGE OF OSTEOPATHIC MEDICINE, Blacksburg, VA (US); VIRGINIA TECH INTELLECTUAL PROPERTIES, INC., Blacksburg, VA (US)

(72) Inventors: Christopher M. Reilly, Blacksburg, VA (US); Abdul Gafoor, Blacksburg, VA (US); David L. Caudell, Blacksburg, VA (US); Nicole L. Regna, Blacksburg, VA (US); Matthew B. Jarpe, Quincy, MA (US)

(73) Assignees: ACETYLON PHARMACEUTICALS, INC., Boston, MA (US); EDWARD VIA COLLEGE OF OSTEOPATHIC MEDICINE, Blacksburg, VA (US); VIRGINIA TECH INTELLECTUAL PROPERTIES, INC., Blacksburg, VA (US)

(21) Appl. No.: 15/023,035

(22) PCT Filed: Sep. 19, 2014
(86) PCT No.: PCT/US2014/056584

§ 371 Date: Mar. 18, 2016

Related U.S. Application Data
(60) Provisional application No. 61/880,427, filed on Sep. 20, 2013.

(51) Int. Cl. A61K 31/505

(52) U.S. Cl. CPC A61K 31/505

(57) ABSTRACT

An HDAC6 inhibitor (a compound of Formula I) is shown to reduce the pathogenesis associated with the B cell mediated autoimmune disease, systemic lupus erythematosus (SLE) Administration of a compound of Formula I attenuated many of the symptoms characteristic of SLE including splenomegaly, abnormal B cell differentiation, an increase in the number double-negative thymic T cells, an increase in the level of auto-antibodies such as anti-dsDNA, immune complex-mediated glomerulonephritis and an increase in inflammatory cytokine production. Treatment with a compound of Formula I also increased the number of the subject's splenic Treg cells while removing circulating auto-antibodies Inhibition of HDAC6 altered bone marrow B cell differentiation by increasing the percentage of cells in the early-stage developmental fractions of both pro- and pre-B cells. These results demonstrate HDAC6 inhibition with a compound of Formula I can treat SLE disease by altering aberrant T and B cell differentiation.

### TECHNICAL FIELD

This disclosure reports on the administration of HDAC6 inhibitors for the treatment of disorders caused by abnormal lymphocyte function.

### BACKGROUND

Post-translational modification of proteins through acetylation and deacetylation of lysine residues plays a critical role in regulating a variety of cellular functions, including the control of cell shape, differentiation and proliferation. Histone deacetylases (HDACs) are zinc-binding hydrolases that catalyze the deacetylation of lysine residues on histones as well as non-histone proteins (Haberland et al Nature Rev. Genet. 2009, 10, 32-42). Eleven Zn binding human HDACs have been identified (Taunton et al. Science 1996, 272, 408-411; Yang et al. J. Biol. Chem. 1997, 272, 28001-28007; Grozinger et al. Proc. Natl. Acad. Sd. U.S.A. 1999, 96, 4868-4873; Kao et al. Genes Dev. 2000, 14, 55-66. Hu et al J. Biol. Chem. 2000, 275, 15254-15264; Zhou et al. Proc. Natl. Acad. Sci U.S.A. 2001, 98, 10572-10577; Venter et al. Science 2001, 291, 1304-1351). These members are classified into four families: Class I (HDAC1, 2 and 3), Class IIa (HDAC4, 5, 7 and 9), Class IIb (HDAC6 and 10) and Class IV (HDAC11).

Class I HDACs (HDACs 1, 2 and 3) modulate gene expression through deacetylation of the N-acetyl-lysine residues of histone proteins and other transcriptional regulators in the nucleus of the cell (Hassig et al. Curr. Opin. Chem. Biol. 1997, 1, 300-308).

HDAC6, a class IIb HDAC, is unique amongst the zinc dependent HDACs in humans. Located in the cytoplasm, HDAC6 has two catalytic domains and an ubiquitin binding domain in its C terminal region. The substrates of HDAC 6 include tubulin, peroxiredoxin, cortactin and heat shock protein 90 (hsp90) but not histones. HDAC6 plays a key role in microtubule dynamics including cell migration and cell-cell interactions and it is required for aggresome formation with ubiquitinated proteins.

Provided herein are methods of using small molecule inhibitors of HDAC6 and pharmaceutical compositions thereof to treat diseases resulting from abnormal lymphocyte function.

### SUMMARY OF THE INVENTION

This disclosure provides for methods of treating diseases caused by abnormal lymphocyte function with a compound of Formula I.

In one embodiment, the disclosure provides for a method of treating abnormal lymphocyte function in a subject comprising:

administering a therapeutically effective amount of a compound of formula I:

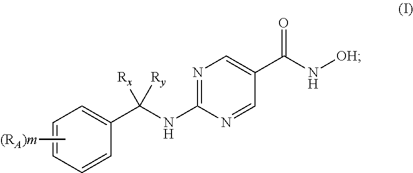

or a pharmaceutically acceptable salt, ester or prodrug thereof to a subject,

wherein the amount of the compound of Formula I is effective at treating the subject's abnormal lymphocyte function.

In one embodiment, the abnormal lymphocyte function comprises a defect in apoptosis.

In one embodiment, the abnormal lymphocyte function can impair lymphocyte development.

In another embodiment, the impaired lymphocyte function can lead to an increase the number of immature lymphocytes.

In another embodiment, the impaired lymphocyte function can produce an auto-reactive lymphocyte.

In one embodiment, the abnormal lymphocyte function results in a B cell mediated autoimmune disease, for example, systemic lupus erythematosus (SLE).

In another embodiment, the compound of Formula I can be ACY-738, wherein ACY-738 has the structure:

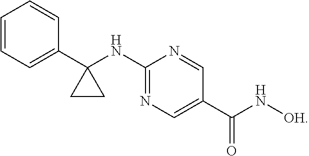 [SMILES: O=C(NO)c1cnc(NC2(c3ccccc3)CC2)nc1]

In another embodiment, a therapeutically effective amount of ACY-738 comprises about 20 mg/kg to about 5 mg/kg of ACY-738.

The subject can be a human.

In other embodiments, the administration of the compound of Formula I reduces a subject's splenomegaly, aberrant B cell differentiation, the increase in the subject's double-negative thymic T cells, sera anti-dsDNA levels, immune complex-mediated glomerulonephritis or inflammatory cytokine production.

In yet another embodiment, the administration of the compound of Formula I reduces the number of the subject's autoreactive B cells.

The disclosure further provides for a method for decreasing the pathogenesis associated with a B cell mediated autoimmune disease comprising:

administering to a subject in need thereof a therapeutically effective amount of a compound of formula I:

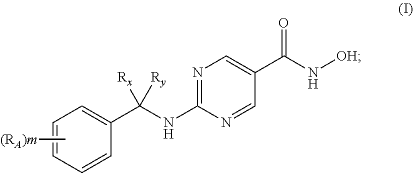

or a pharmaceutically acceptable salt, ester or prodrug thereof,

wherein the amount of the compound of formula I is effective at reducing the pathogenesis associated with a B cell mediated autoimmune disease, for example systemic lupus erythematosus.

In one embodiment, the compound of Formula I is ACY-738, wherein ACY-738 has the structure:

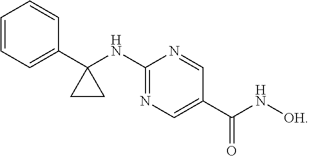 [SMILES: O=C(NO)c1cnc(NC2(c3ccccc3)CC2)nc1]

The disclosure further provides for a kit comprising a therapeutically effective amount of a compound of formula I and instructions for use in treating a B cell mediated autoimmune disease, e.g. systemic lupus erythematosus.

### BRIEF DESCRIPTION OF THE DRAWINGS

FIG. 1 depicts the levels of pro- and pre-B cells in NZB/W mice. Bone marrow (BM) cells were harvested from pre-diseased and diseased NZB/W mice. After labeling with fluorescently tagged antibodies specific for pro- and pre-B cells, pro-B cell (B220+CD43+) and pre-B cell (B220+CD43−) populations were analyzed by flow cytometry (n=3; *p<0.05, ** p<0.005, ***p<0.0005).

FIG. 2 shows HDAC6 inhibition alters pro- and pre-B cell populations in the bone marrow. Harvested bone marrow cells from ACY-738 treated NZB/W mice were stained with B220 and CD43. Pro-B cell (B220+CD43+) and pre-B cell (B220+CD43−) populations were then analyzed by flow cytometry (n≧5; *p<0.05, ** p<0.005, ***p<0.0005).

FIG. 3 shows that the treatment of NZB/W mice with ACY-738 had no effect on the percentage of splenic or peripheral B cells at either the low or high dose (n≧5).

FIG. 4 shows the percentage of double negative (DN) thymic T cells is reduced following treatment of NZB/W mice with ACY-738.

FIG. 5 shows specific HDAC6 inhibition increases the Treg phenotype in NZB/W mice (n≧5; *p<0.05).

FIG. 6 shows an assessment on survival rate, body weight, proteinuria, average spleen weight and overall disease progression in NZB/W mice treated with ACY-738 (n≧5; *p<0.05, ** p<0.005).

FIG. 7 shows an evaluation of SLE sera biomarkers of disease in NZBW mice following ACY-738 therapy (n≧5; *p<0.05, ** p<0.005).

FIG. 8 shows the levels of cytokine production in NZBW mice following ACY-738 therapy (n≧5; *p<0.05, *** p<0.0005).

FIG. 9 shows glomerular IL-6, IL-10, and TGF-β mRNA levels in NZBW mice following ACY-738 therapy (n≧5; *p<0.05, ** p<0.005, ***p<0.0005).

FIG. 10 shows HDAC6 inhibition decreased glomerular immune complex deposition (n≧5; *p<0.05, ** p<0.005).

FIG. 11 shows SLE-associated renal pathology was decreased following ACY-738 therapy (n≧5; *p<0.05).

### DETAILED DESCRIPTION OF THE INVENTION

### Definitions

Listed below are definitions of various terms used to describe this invention. These definitions apply to the terms as they are used throughout this specification and claims, unless otherwise limited in specific instances, either individually or as part of a larger group.

The number of carbon atoms in a hydrocarbyl substituent can be indicated by the prefix “Cₓ-C_{y},” where x is the minimum and y is the maximum number of carbon atoms in the substituent. Likewise, a Cₓ chain means a hydrocarbyl chain containing x carbon atoms.

The term “alkyl,” as used herein, refers to saturated, straight- or branched-chain hydrocarbon moieties containing, in certain embodiments, between one and six, or one and eight carbon atoms, respectively. Examples of C₁-C₆ alkyl moieties include, but are not limited to, methyl, ethyl, propyl, isopropyl, n-butyl, tert-butyl, neopentyl, n-hexyl moieties; and examples of C₁-C₈ alkyl moieties include, but are not limited to, methyl, ethyl, propyl, isopropyl, n-butyl, tert-butyl, neopentyl, n-hexyl, heptyl, and octyl moieties.

The term “alkenyl,” as used herein, denotes a monovalent group derived from a hydrocarbon moiety containing, in certain embodiments, from two to six, or two to eight carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “alkynyl,” as used herein, denotes a monovalent group derived from a hydrocarbon moiety containing, in certain embodiments, from two to six, or two to eight carbon atoms having at least one carbon-carbon triple bond. The alkynyl group may or may not be the point of attachment to another group. Representative alkynyl groups include, but are not limited to, for example, ethynyl, 1-propynyl, 1-butynyl, heptynyl, octynyl and the like.

The term “alkoxy” refers to an —O-alkyl moiety.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like.

The term “aralkyl,” or “arylalkyl,” as used herein, refers to an alkyl residue attached to an aryl ring. Examples include, but are not limited to, benzyl, phenethyl and the like.

The term “carbocyclic,” as used herein, denotes a monovalent group derived from a monocyclic or polycyclic saturated, partially unsatured, or fully unsaturated carbocyclic ring compound. Examples of carbocyclic groups include groups found in the cycloalkyl definition and aryl definition.

The term “cycloalkyl,” as used herein, denotes a monovalent group derived from a monocyclic or polycyclic saturated or partially unsatured carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2]octyl. Also contemplated are monovalent groups derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, moieties or ring system having at least one aromatic ring, having from five to ten ring atoms of which one ring atom is selected from S, O and N; zero, one or two ring atoms are additional heteroatoms independently selected from S, O and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl,” as used herein, refers to an alkyl residue attached to a heteroaryl ring. Examples include, but are not limited to, pyridinylmethyl, pyrimidinylethyl and the like.

The term “heterocycloalkyl,” as used herein, refers to a non-aromatic 3-, 4-, 5-, 6- or 7-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “alkylamino” refers to a group having the structure —NH(C₁-C₁₂ alkyl) where C₁-C₁₂ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

In accordance with the invention, any of the aryls, substituted aryls, heteroaryls and substituted heteroaryls described herein, can be any aromatic group. Aromatic groups can be substituted or unsubstituted.

The terms “hal,” “halo” and “halogen,” as used herein, refer to an atom selected from fluorine, chlorine, bromine and iodine.

The term “oxo” as used herein, refers to oxygen that is attached to a carbon, preferably by a double bond (e.g., carbonyl).

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to:

alkyl, alkenyl, alkynyl, cycloalkyl, aryl, heterocycloalkyl, heteroaryl, arylalkyl, heteroarylalkyl, haloalkyl (e.g., —CF₃), haloalkoxy (e.g., —OCF₃),

—F, —Cl, —Br, —I,

—OH, protected hydroxy, oxygen, oxo,

—NO₂, —CN,

—NH₂, protected amino, —NH—C₁-C₁₂-alkyl, —NH-aryl, -dialkylamino,

—O—C₁-C₁₂-alkyl, —O-aryl,

—C(O)—, —C(O)O—, —C(O)NH—, —OC(O)—, —OC(O)O—, —OC(O)NH—, —NHC(O)—, —NHC(O)O—,

—C(O)—C₁-C₁₂-alkyl, —C(O)—C₃-C₁₂-cycloalkyl, —C(O)-aryl, —C(O)-heteroaryl, —C(O)— heterocycloalkyl,

—C(O)O—C₁-C₁₂-alkyl, —C(O)O—C₃-C₁₂-cycloalkyl, —C(O)O-aryl, —C(O)O-heteroaryl, —C(O)O-heterocycloalkyl,

—CONH₂, —CONH—C₁-C₁₂-alkyl, —CONH-aryl,

—OCO₂—C₁-C₁₂-alkyl, —OCO₂-aryl, —OCONH₂, —OCONH—C₁-C₁₂-alkyl, —OCONH-aryl,

—NHC(O)—C₁-C₁₂-alkyl, —NHC(O)-aryl, —NHCO₂—C₁-C₁₂-alkyl, —NHCO₂— aryl,

—S(O)—C₁-C₁₂-alkyl, —S(O)-aryl, —SO₂NH—C₁-C₁₂-alkyl, —SO₂NH— aryl,

—NHSO₂—C₁-C₁₂-alkyl, —NHSO₂-aryl, or

—SH, —S—C₁-C₁₂-alkyl, or —S-aryl.

In certain embodiments, the optionally substituted groups include the following: C₁-C₁₂-alkyl, C₂-C₁₂-alkenyl, C₂-C₁₂-alkynyl, C₃-C₁₂-cycloalkyl, C₃-C₁₂-aryl, C₃-C₁₂-heterocycloalkyl, C₃-C₁₂-heteroaryl, C₄-C₁₂-arylalkyl, or C₂-C₁₂-heteroarylalkyl.

It is understood that the aryls, heteroaryls, alkyls, and the like can be further substituted.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

A subject can also refer to animal models of an auto immune disease, such as SLE. Spontaneous SLE animal models include the F1 hybrid between the New Zealand Black (NZB) and New Zealand White (NZW) strains (NZB/W F1) and its derivatives as well as the MRL/lpr, and BXSB/Yaa strains. Induced LEP animal models include heavy metal induced autoimmunity, the pristine TMPD-induced model, drug induced lupus and the chronic graft-versus-host-disease models (cGVHD). All of these models portray their own iterations of lupus-like diseases with a subset of symptoms akin to those observed in human SLE, namely, autoantibody production, lymphoid activation and hyperplasia, and lupus nephritis. Other animal models of SLE are described in U.S. Pat. No. 7,265,261.

Treat”, “treating” and “treatment” can refer to a method of alleviating or abating a disease resulting from an abnormal lymphocyte function. In certain embodiments, the autoimmune disease can be systemic lupus erythematosus (SLE).

In another embodiment, the terms “treating” or “treatment” can refer to any improvement in one or more clinical symptoms of an abnormal lymphocyte function.

A symptom of abnormal lymphocyte function can include splenomegaly, aberrant B cell differentiation, an increase in the number double-negative thymic T cells, an increase in the level of anti-dsDNA, immune complex-mediated glomerulonephritis, an increase in inflammatory cytokine production, an increase in the number of a subject's splenic Treg cells, an increase in the number of autoantibodies or an increase in the percentage of the subject's cells in the early-stage developmental fractions of both pro- and pre-B cells.

In other embodiments, an improvement of a symptom of abnormal lymphocyte function includes, but is not limited to, decreased joint pain, swelling and redness, low grade fever, skin rashes, vasculitis, fatigue, loss of appetite, nausea, and weight loss, chest pain, bruising, menstrual irregularities, sleep disorders, such as restless legs syndrome and sleep apnea, dryness of the eyes and mouth, brittle hair or hair loss, increase in the remission period between acute disease attacks; decrease in the time length of the acute attack; prevention of the onset of severe disease, etc. An improvement of a symptom of abnormal lymphocyte function can also include renal functions (decrease in blood urea, creatinine, or proteinuria).

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17.sup.th ed., Mack Publishing Company, Easton, Pa., 1985, p. 1418 and Journal of Pharmaceutical Science, 66, 2 (1977), each of which is incorporated herein by reference in its entirety.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

As used herein, the term “abnormal lymphocyte function” refers to one or more defects in lymphocyte function that can be treated by inhibiting an activity of HDAC6.

A “defect in lymphocyte function” can include, but is not limited to, dysregulation of lymphocyte proliferation, abnormal signal transduction or secretion of cytokine and/or impaired B or T lymphocyte differentiation.

In other embodiments, a defect in lymphocyte function can refer to impaired apoptosis during lymphocyte differentiation.

In another embodiment, abnormal lymphocyte function can lead to the production of auto-reactive lymphocytes.

As used herein, an “auto-reactive lymphocyte” can refer to a B or T lymphocyte that reacts to auto-antigens.

In another embodiment, abnormal lymphocyte function can lead to the production of autoantibodies by B lymphocytes.

In another embodiment, abnormal lymphocyte function can cause an autoimmune disease.

As used herein, an “autoimmune disease” can include, but is not limited to, acute disseminated encephalomyelitis, Addison's disease, agammaglobulinemia, alopecia areata, amyotrophic lateral sclerosis (also Lou Gehrig's disease; motor neuron disease), ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria autoimmune uveitis, balo disease/balo concentric sclerosis, Basedow's disease, Behcet's disease, Berger's disease Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, crest syndrome, Crohn's disease (one of two types of idiopathic inflammatory bowel disease “ibd”), Cushing's syndrome, cutaneous leukocytoclastic angiitis, dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, evan's syndrome, fibrodysplasia ossificans progressiva, fibrosing alveolitis (or idiopathic pulmonary fibrosis), gastritis, gastrointestinal pemphigoid, glomerulonephritis, Goodpasture's syndrome, Graves' disease, Guillain-Barré syndrome (gbs), Hashimoto's encephalopathy, Hashimoto's thyroiditis, Henoch-Schonlein purpura, herpes gestationis aka gestational pemphigoid, Goodpasture's syndrome, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura (see autoimmune thrombocytopenic purpura), iga nephropathy, inclusion body myositis, chronic inflammatory demyelinating polyneuropathy, interstitial cystitis, juvenile idiopathic arthritis aka juvenile rheumatoid arthritis, Kawasaki's disease, Lambert-Eaton myasthenic syndrome, leukocytoclastic vasculitis, lichen planus, lichen sclerosus, linear iga disease, lupoid hepatitis aka autoimmune hepatitis, lupus erythematosus, Majeed syndrome, Méniere's disease, microscopic polyangiitis, Miller-Fisher syndrome, mixed connective tissue disease, morphea, Mucha-Habermann disease aka pityriasis lichenoides et varioliformis acuta, multiple sclerosis, myasthenia gravis, myositis, narcolepsy, neuromyelitis optica (also Devic's disease), neuromyotonia, occular cicatricial pemphigoid, opsoclonus myoclonus syndrome, Ord's thyroiditis, palindromic rheumatism, pandas (pediatric autoimmune neuropsychiatric disorders associated with streptococcus), paraneoplastic cerebellar degeneration, paroxysmal nocturnal hemoglobinuria (pnh), Parry Romberg syndrome, Parsonage-Turner syndrome, pars planitis, pemphigus vulgaris, pernicious anaemia, perivenous encephalomyelitis, Poems syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome, Schnitzler syndrome, scleritis, scleroderma, serum sickness, Sjögren's syndrome, spondyloarthropathy, Still's disease, Stiff person syndrome, subacute bacterial endocarditis, Susac's syndrome, Sweet's syndrome, Sydenham chorea, sympathetic ophthalmia, systemic lupus erythematosus, Takayasu's arteritis, temporal arteritis (also known as “giant cell arteritis”), thrombocytopenia, Tolosa-Hunt syndrome, transverse myelitis, ulcerative colitis (one of two types of idiopathic inflammatory bowel disease “ibd”), undifferentiated connective tissue disease, undifferentiated spondyloarthropathy, urticarial vasculitis, vasculitis, vitiligo or Wegener's granulomatosis.

As used herein, HDAC6 inhibition refers to the inhibition of an activity of HDAC6. In certain embodiments, HDAC6 inhibition refers to the inhibition of an activity of HDAC6 by a compound of Formula I as described below.

### Compounds of the Invention

Provided herein is a compound of formula I:

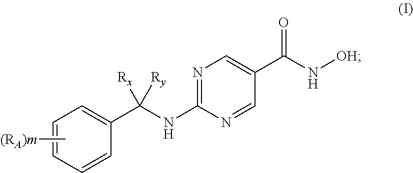

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

Representative compounds of Formula I include, but are not limited to, the following compounds of Table 1 below, or pharmaceutically acceptable salts, esters or prodrugs thereof

TABLE 1

| | |
|---|---|
| 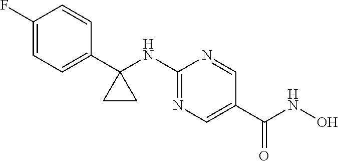 [SMILES: O=C(NO)c1cnc(NC2(c3ccc(F)cc3)CC2)nc1] | 81 |
| 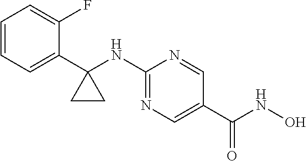 [SMILES: O=C(NO)c1cnc(NC2(c3ccccc3F)CC2)nc1] | 82 |
| 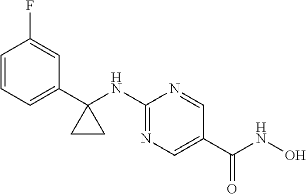 [SMILES: O=C(NO)c1cnc(NC2(c3cccc(F)c3)CC2)nc1] | 87 |
| 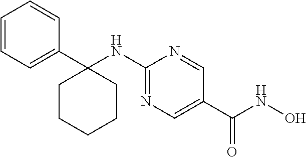 [SMILES: O=C(NO)c1cnc(NC2(c3ccccc3)CCCCC2)nc1] | 84 |
| 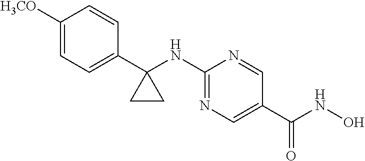 [SMILES: COc1ccc(C2(Nc3ncc(C(=O)NO)cn3)CC2)cc1] | 97 |
| 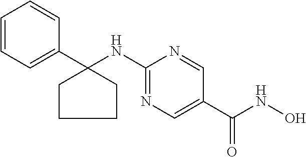 [SMILES: O=C(NO)c1cnc(NC2(c3ccccc3)CCCC2)nc1] | 88 |
| 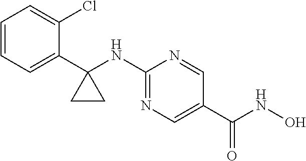 [SMILES: O=C(NO)c1cnc(NC2(c3ccccc3Cl)CC2)nc1] | 90 |
| 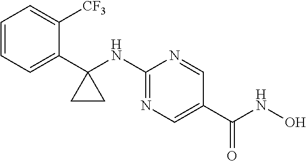 [SMILES: O=C(NO)c1cnc(NC2(c3ccccc3C(F)(F)F)CC2)nc1] | 91 |
| 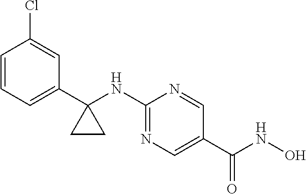 [SMILES: O=C(NO)c1cnc(NC2(c3cccc(Cl)c3)CC2)nc1] | 94 |
| 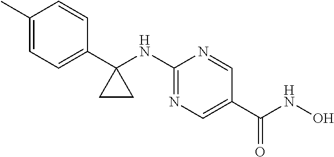 [SMILES: Cc1ccc(C2(Nc3ncc(C(=O)NO)cn3)CC2)cc1] | 93 |
| 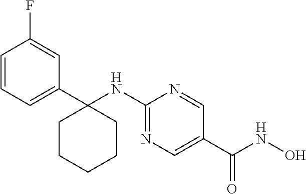 [SMILES: O=C(NO)c1cnc(NC2(c3cccc(F)c3)CCCCC2)nc1] | 101 |
| 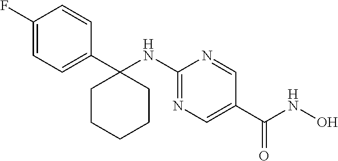 [SMILES: O=C(NO)c1cnc(NC2(c3ccc(F)cc3)CCCCC2)nc1] | 100 |
| 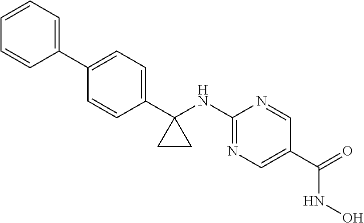 [SMILES: O=C(NO)c1cnc(NC2(c3ccc(-c4ccccc4)cc3)CC2)nc1] | 117 |

In certain embodiments, the compound of the invention is selected from Table 2, or pharmaceutically acceptable salts, esters or prodrugs thereof:

TABLE 2

| | |
|---|---|
| 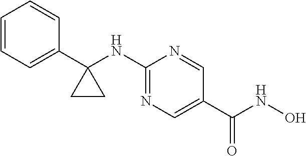 [SMILES: O=C(NO)c1cnc(NC2(c3ccccc3)CC2)nc1] | 73 |
| 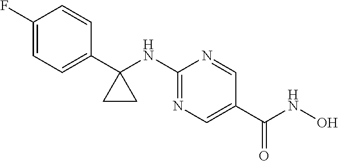 [SMILES: O=C(NO)c1cnc(NC2(c3ccc(F)cc3)CC2)nc1] | 81 |
| 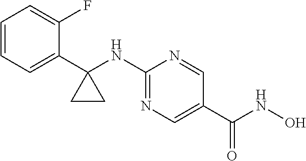 [SMILES: O=C(NO)c1cnc(NC2(c3ccccc3F)CC2)nc1] | 82 |
| 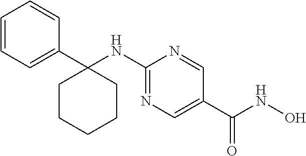 [SMILES: O=C(NO)c1cnc(NC2(c3ccccc3)CCCCC2)nc1] | 84 |
| 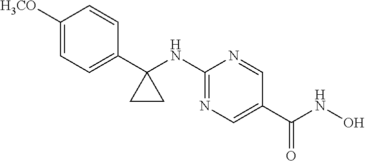 [SMILES: COc1ccc(C2(Nc3ncc(C(=O)NO)cn3)CC2)cc1] | 97 |
| 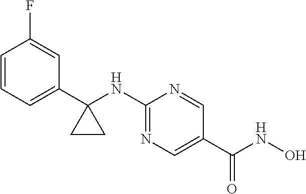 [SMILES: O=C(NO)c1cnc(NC2(c3cccc(F)c3)CC2)nc1] | 87 |
| 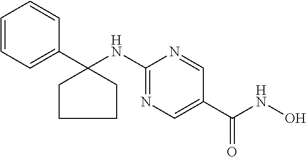 [SMILES: O=C(NO)c1cnc(NC2(c3ccccc3)CCCC2)nc1] | 88 |
| 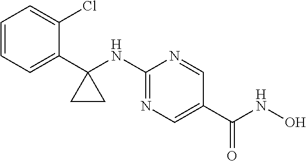 [SMILES: O=C(NO)c1cnc(NC2(c3ccccc3Cl)CC2)nc1] | 90 |
| 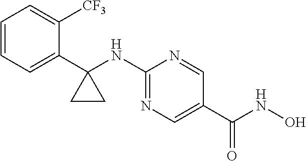 [SMILES: O=C(NO)c1cnc(NC2(c3ccccc3C(F)(F)F)CC2)nc1] | 91 |
| 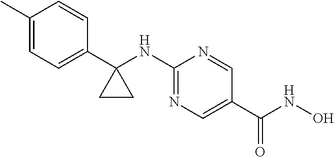 [SMILES: Cc1ccc(C2(Nc3ncc(C(=O)NO)cn3)CC2)cc1] | 93 |
| 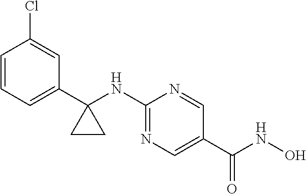 [SMILES: O=C(NO)c1cnc(NC2(c3cccc(Cl)c3)CC2)nc1] | 94 |

In a particular embodiment, the compound of Formula I is the compound 73 (also referred herein as ACY-738), or a pharmaceutically acceptable salt, ester or prodrug thereof:

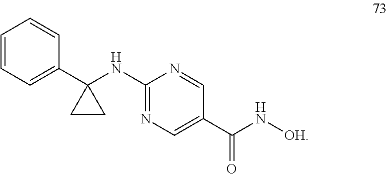 [SMILES: O=C(NO)c1cnc(NC2(c3ccccc3)CC2)nc1]

In another particular embodiment, the compound of Formula I is the compound 101 (also referred herein as ACY-775), or a pharmaceutically acceptable salt, ester or prodrug thereof:

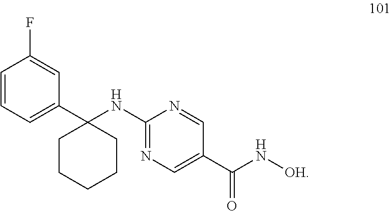 [SMILES: O=C(NO)c1cnc(NC2(c3cccc(F)c3)CCCCC2)nc1]

In another aspect, the invention provides a pharmaceutical composition comprising a compound of formula I:

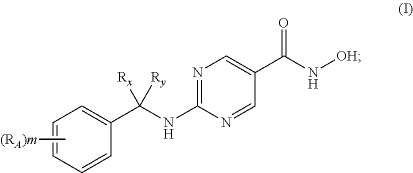

or a pharmaceutically acceptable salt, ester or prodrug thereof,
wherein,
Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

The compositions and pharmaceutical compositions provided herein can be used to treat a subject's abnormal lymphocyte function.

In certain embodiments, the abnormal lymphocyte function comprises a defect in apoptosis or an impairment of lymphocyte development.

In one embodiment, the abnormal lymphocyte function can lead to an increase the number of immature lymphocytes.

In one embodiment, the abnormal lymphocyte function can produce an autoreactive lymphocyte.

In another embodiment, the compositions and pharmaceutical compositions provided herein can be used to treat an abnormal lymphocyte function that results in a B cell mediated autoimmune disease, for example, systemic lupus erythematosus (SLE).

In another embodiment, the compositions and pharmaceutical compositions provided herein can reduce a subject's splenomegaly, aberrant B cell differentiation, the increase in the subject's double-negative thymic T cells, sera anti-dsDNA levels, immune complex-mediated glomerulonephritis or inflammatory cytokine production.

In yet another embodiment, the compositions and pharmaceutical compositions provided herein can be used to reduce the number of a subject's autoreactive B cells.

Another object of the present invention is the use of a compound as described herein in the manufacture of a medicament for use in the treatment of a disorder or disease herein. Another object of the present invention is the use of a compound as described herein for use in the treatment of a disorder or disease herein.

### Synthesis of the Compounds of the Invention

The synthesis of the compounds of the invention can be found below.

Another embodiment is a method of making a compound of formula I using any one, or combination of, reactions delineated herein. The method can include the use of one or more intermediates or chemical reagents delineated herein.

Another aspect is an isotopically labeled compound of formula I delineated herein. Such compounds have one or more isotope atoms which may or may not be radioactive (e.g., ³H, ²H, ¹⁴C, ¹³C, ³⁵S, ³²P, ¹²⁵I, and ¹³¹I) introduced into the compound. Such compounds are useful for drug metabolism studies and diagnostics, as well as therapeutic applications.

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999, and subsequent editions thereof.

Compounds of the present invention can be conveniently prepared or formed during the process of the invention, as solvates (e.g., hydrates). Hydrates of compounds of the present invention can be conveniently prepared by recrystallization from an aqueous/organic solvent mixture, using organic solvents such as dioxan, tetrahydrofuran or methanol.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included in the present invention. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds of this invention may also be represented in multiple tautomeric forms, in such instances, the invention expressly includes all tautomeric forms of the compounds described herein. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present invention. All crystal forms of the compounds described herein are expressly included in the present invention.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In embodiments, the invention provides for the intermediate compounds of the formulae delineated herein and methods of converting such compounds to compounds of the formulae herein (e.g., in schemes herein) comprising reacting a compound herein with one or more reagents in one or more chemical transformations (including those provided herein) to thereby provide the compound of any of the formulae herein or an intermediate compound thereof.

The synthetic methods described herein may also additionally include steps, either before or after any of the steps described in any scheme, to add or remove suitable protecting groups in order to ultimately allow synthesis of the compound of the formulae described herein. The methods delineated herein contemplate converting compounds of one formula to compounds of another formula (e.g., in Scheme A, A1 to A2; A2 to A3; A1 to A3). The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, sublimation, crystallization, trituration, solid phase extraction, and chromatography).

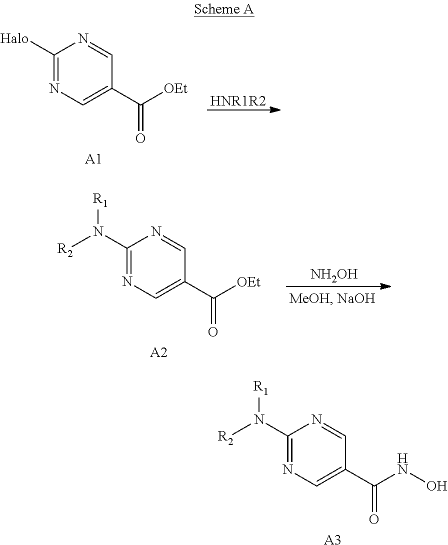

The compounds of this invention may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof. The recitation of an embodiment for a variable herein includes that embodiment as any single embodiment or in combination with any other embodiments or portions thereof.

### Methods of the Invention

In one aspect, the compound of Formula I is administered to a subject for the treatment of a disease or a symptom of a disease caused by abnormal lymphocyte function.

In certain embodiments, a disease caused by abnormal lymphocyte function can be an autoimmune disease including, but not limited to, rheumatoid arthritis, thyroiditis, Hashimoto's thyroiditis, Evans syndrome, multiple sclerosis, myasthenia gravis, type I diabetes uveitis, juvenile-onset or recent-onset diabetes mellitus, uveitis, Graves' disease, psoriasis, sarcoidosis, atopic dermatitis, Crohn's disease, ulcerative colitis, vasculitis, auto-antibody mediated diseases, aplastic anemia, Evan's syndrome or autoimmune hemolytic anemia.

In another embodiment, a disease caused by abnormal lymphocyte function can be a lymphoproliferative disorder, including, but not limited to, lymphocytosis, follicular lymphoma, chronic lymphocytic leukemia (CLL), acute lymphoblastic leukemia (ALL), hairy cell leukemia, lymphomas, diffuse large B cell lymphoma, follicular lymphoma, MALT lymphoma, Burkitt's B cell or T cell lymphoma, Mycosis fungoides, T cell lymphomas, Hodgkin's lymphoma, Non-Hodgkin's lymphoma, thymic lymphoma, multiple myeloma, Waldenstrom's macroglobulinemia, Wiskott-Aldrich syndrome, post-transplant lymphoproliferative disorder, X-linked lymphoproliferative disorder or autoimmune lymphoproliferative syndrome (ALPS).

In a preferred embodiment, the abnormal lymphocyte function causes systemic lupus erythematosus (SLE).

In another aspect, a therapeutically effective amount of a compound of Formula I is injected intraperitoneally into a subject with systemic lupus erythematosus. Body weight, proteinuria, sera anti-dsDNA, Ig isotypes, and cytokine levels are measured and kidney disease is determined using sera and urinary markers of SLE. Flow cytometric analysis is used to assess thymic and splenic T cell profiles as well as bone marrow, splenic, and peripheral B cell differentiation patterns.

In another aspect, a therapeutically effective amount of a compound of Formula I is injected intraperitoneally into a subject with a lymphoproliferative disorder, wherein the compound of Formula I treats the lymphoproliferative disorder.

In another aspect, a therapeutically effective amount of a compound of Formula I is injected intraperitoneally into a subject with lymphoma, wherein the compound of Formula I treats the lymphoma.

HDAC6 inhibition with a compound of Formula I can also decrease many hallmarks of SLE disease including splenomegaly, immune complex-mediated glomerulonephritis, sera anti-dsDNA levels, and inflammatory cytokine production. The number of double-negative thymic T cells also decreases whereas the percentage of splenic Treg cells increases. HDAC6 inhibition also affects bone marrow B cell differentiation by increasing the percentage of cells in the early-stage developmental fractions of both pro- and pre-B cells.

In a preferred embodiment, the compound of Formula I can be ASY-738.

### HDAC6 Inhibition Corrects a Defect in One or More of the Developmental Checkpoints During Bone Marrow B Cell Development.

In normal subjects, B cells that develop auto-reactive B cell receptors (BCRs) are removed by both positive and negative selection from the bone marrow in three ways—by receptor editing, deletion, and anergy (Hardy et al Annual review of Immunology 2001, 19:595-621; Dorner et al. Arthritis research & therapy 2009, 11(5):247). It is estimated approximately 55-75% of the repertoire produced by Ig gene rearrangement in the bone marrow is auto-reactive (Yurasov et al. The Journal of Experimental Medicine 2005, 201(5):703-711). These autoreactive B cells are removed at two checkpoints (Domer et al. Arthritis research & therapy 2011, 13(5):243). The majority of auto-reactive B cells are removed in the bone marrow while the cells are still immature (Lu et al. Journal of Immunology 1999, 162(4):1931-1940). V(D)J and class switch recombination in the bone marrow produce DNA double strand breaks that result in apoptosis of auto-reactive B cells (Hardy et al. Annual review of immunology 2001, 19:595-621). The apoptotic index and the apoptotic rate, for the removal of auto-reactive B cells, are greatest around the pro/pre-B cell transition (Lu et al. Immunological reviews 2000, 175:158-174).

While there is no difference in the overall number of pro- or pre-B cells between pre-diseased and SLE diseased subjects, the proportions of cells in the pro- and pre-B cell developmental fractions are altered as a result of SLE. SLE subjects have decreased numbers of pro-B cells in the late C and C′ fractions and in the early pre-B cell fractions D and E when compared to normal subjects. There is also a notable increase in the percentage of B cells in the late pre-B stage, fraction F. Bone marrow B cells in subjects with SLE therefore undergo differentiation at an accelerated rate with defective regulation of B cell checkpoints, resulting in failure to remove defective B cells. SLE therefore appears to be caused by an apoptotic defect in bone marrow differentiation at the pre-B/pro-B stages.

While treatment with a compound of Formula I has no effect on the total numbers of pro-B cells, there is a significant decrease in the percentage of total pre-B cells. HDAC6 inhibition increases the percentage of early-stage pro-B cells in fraction A and the number of cells in the early pre-B cell developmental stages D and E. Furthermore treatment with a compound of Formula I decreases the percentage of late-stage pre-B cells in fraction F. Thus, HDAC6 inhibition decreases the percentage of cells that develop into immature B cells coupled with a shift from late-stage subsets to early-stage pro- and pre-B cell subsets. HDAC6 inhibition has the greatest effect on the pro- and pre B cell populations inferring the compound of Formula I treats SLE by correcting the apoptotic defect that is present in SLE.

HDAC6 Inhibition does not Change the Distribution of B Cell Populations in the Periphery or in the Spleen

HDAC6 inhibition does not affect the percentages of B cells in the periphery or the distribution of B cells in the splenic developmental stages. In normal subjects, after immature B cells leave the bone marrow, they continue to develop in secondary lymphoid organs including the spleen. As immature B cells develop into mature B cells they become antigen-specific. Thus, B cells that escape negative selection during bone marrow differentiation may mature in the spleen to become marginal zone or follicular cells. IgM′ IgD′ B cells leave the bone marrow as transitional cells and enter the spleen where they mature into follicular B cells or marginal-zone B cells Inhibition of HDAC6 did not alter the percentages of B cells in transitional, follicular or marginal zone stages suggesting that the HDAC6 inhibitor is acting during early B cell development in the bone marrow and not on peripheral or splenic B cells. This further confirms that HDAC6 inhibition decreases SLE by restoring the function of a checkpoint during bone marrow B cell development.

HDAC6 Inhibition Removes Auto-Reactive B Cells that Produce Autoantibodies.

In subjects with SLE, auto-antibody production, including anti-dsDNA antibodies (Mok et al. Journal of Clinical Pathology 2003, 56(7):481-490), increases and results in a corresponding increase in proteinuria and glomerulonephritis. The IgG autoantibodies produced in SLE individuals form immune complexes that become lodged in renal glomeruli, resulting in increased activation of the immune system and inflammation. The damaged kidneys are then unable to properly filter proteins, which pass into the urine and cause elevated levels of proteinuria. Thus the decrease in IgG production in the sera following HDAC6 inhibition, correlates with reduced glomerular immune complex deposition, SLE-associated kidney pathology, and proteinuria. This indicates that although HDAC6 inhibition doesn't affect the numbers of peripheral B cells, it does alter mature plasma cell production of autoantibodies. HDAC6 inhibition therefore removes auto-reactive B cells that produce autoantibodies.

### HDAC6 Inhibition Increases the Treg Phenotype

In subjects with SLE, the number of Treg cells are substantially reduced. This leads to a concomitant increase in antibody production. Treatment of SLE with the compound of Formula I reverses this trend by increasing the number of Treg cells and reducing the levels of autoantibody production. Moreover, pan-HDAC inhibitors, but not class I specific HDAC inhibitors, increase populations of Treg cells suggesting that the Treg profile may be regulated directly by a class IIb HDAC, such as HDAC6.

Naïve CD4+ T cells differentiate into Treg cells following TGF-β stimulation, which promotes Foxp3 expression. In subjects with SLE, sera TGF-β levels are reduced. Treatment of SLE with a compound of Formula I mitigates the reduction of TGF-β in a dose-dependent manner which increases the size of the Treg population. Conversely, glomerular mRNA expression of TGF-β decreases following HDAC6 inhibitor treatment indicating a dual role of TGF-β in SLE pathogenesis. Anti-inflammatory cytokines including TGF-β are produced in order to combat inflammation within target organs such as the kidneys. The increased production of anti-inflammatory cytokines causes deposition of extracellular matrix and fibrosis. Elevated levels of TGF-β in immune cells coincides with a reduction in TGF-β in target organs leading to autoimmune disease. HDAC6 inhibition reverses this trend by increasing the sera levels of TGF-β, while decreasing TGF-β in the glomeruli of the kidneys. HDAC6 inhibition may therefore increase Treg populations through altered TGF-β production.

### HDAC6 Reduces IL-6, IL-10, and IL-1β Cytokine Production in SLE

During, SLE there is an imbalance between the production of Th1 and Th2 cytokines. However, inhibition of HDAC6 reverses the altered IL-6, IL-10, and IL-1β cytokine production trends that are characteristic of SLE. The proinflammatory cytokine IL-6 is upregulated in SLE and contributes to overproduction of IgG. Following treatment with the compound of Formula I, IL-6 is undetectable in the kidneys of SLE subjects. This reduction in IL-6 correlates with a decrease in total IgG and the IgG2a isotype levels in the sera. Similarly, proinflammatory cytokine IL-1β, which plays a significant role in the pathogenicity of a number of autoimmune diseases including SLE, is also diminished by treatment with the compound of Formula I. Hence, HDAC6 inhibition of inflammatory cytokine production in SLE subjects reduces the sera levels of autoantibodies and attenuates the symptoms of SLE.

HDAC6 Inhibition Reduces the Number of Double Negative (DN) T Cells in Subjects with SLE

The number of DN T cells is often elevated in individuals with SLE. HDAC6 inhibition reduces the numbers of DN T cells. Thymocytes undergo both positive and negative selection during development to eliminate self-reactive or non-functional T cells. Development begins with progenitor T cells (CD4−CD8−CD3−) which give rise to DN T cells (CD4−CD8−CD3+). DN T cells then develop into DP (CD4+CD8+CD3+) T cells which differentiate into two subsets of mature SP T cells: helper (CD4+CD8−CD3+) or cytotoxic (CD4−CD8+CD3+) T cells. DP thymocytes undergo positive selection to ensure reactivity and specificity. SP T cells undergo negative selection to eliminate autoreactive T cells. The elevated level of DN T cells associated with SLE is attributed to a defect in apoptosis. Additionally, percentages of SP cells are low in SLE patients, suggesting that DN T cells are resistant to apoptotic mechanisms. DN T cells are believed to contribute to SLE pathogenesis through the induction of Ig and anti-dsDNA autoantibody production. Treatment of SLE with the compound of Formula I decreases the number of DN T cells and autoantibody production suggesting that HDAC6 inhibition can attenuate SLE pathogenesis through the regulation of T cell development.

### Pharmaceutical Compositions

In another aspect, the invention provides a pharmaceutical composition comprising a compound of formula I, or a pharmaceutically acceptable ester, salt, or prodrug thereof, together with a pharmaceutically acceptable carrier. This pharmaceutical composition can be used in the treatment of abnormal lymphocyte function.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of an autoimmune disease such as SLE), or they may achieve different effects (e.g., control of any adverse effects).

In certain embodiments, the pharmaceutical compositions of the present invention further comprise one or more additional therapeutically active ingredients. For purposes of the invention, the term “palliative” refers to treatment that is focused on the relief of symptoms of a disease and/or side effects of a therapeutic regimen, but is not curative. For example, palliative treatment encompasses painkillers, anti-nausea medications, anti-pyretics, and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

The pharmaceutical compositions of the present invention comprise a therapeutically effective amount of a compound of the present invention formulated together with one or more pharmaceutically acceptable carriers.

As used herein, the term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

According to the methods of treatment of the present invention, disorders are treated or prevented in a subject, such as a human or other animal, by administering to the subject a therapeutically effective amount of a compound of the invention, in such amounts and for such time as is necessary to achieve the desired result.

The term “therapeutically effective amount” of a compound of the invention, as used herein, means a sufficient amount of the compound so as to decrease one or more of the symptoms caused by an abnormal lymphocyte function in a subject. For example, the abnormal lymphocyte function can cause symptoms of systemic lupus erythematosus. As is well understood in the medical arts a therapeutically effective amount of a compound of this invention will be at a reasonable benefit/risk ratio applicable to any medical treatment.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in controlled release form. Suitable unit dosage forms for oral administration comprise from ca. 1 to 50 mg active ingredient.

In certain embodiments, a therapeutic amount or dose of the compounds of the present invention may range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of symptoms resulting from abnormal lymphocyte function.

It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Kits

The disclosure herein provides for a kit format which comprises package units having different doses of the compound of Formula I for treating an abnormal lymphocyte function in a subject. In certain embodiments, the compound of Formula I is ACY-738.

The kit may also contain one or more of the following items: instructions for use including prescribing information, dosage information, storage information, and the like as well as sterile saline solution, needles, syringes, catheters and first aid materials such as bandages etc. Kits may include containers of reagents mixed together in suitable proportions for performing the methods described herein. Reagent containers preferably contain reagents in unit quantities that obviate measuring steps when performing the subject methods.

The package label can include, for example, instructions to take the compound of Formula I for the treatment of an autoimmune disease. In another embodiment, the package label includes instructions to treat systemic lupus erythematosus.

Packaged compositions are also provided that comprise a therapeutically effective amount of a compound of Formula I, e.g. ACY-738, and a pharmaceutically acceptable carrier or diluent as well as instructions on how to treat an autoimmune disorder such as systemic lupus erythematosis.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

### EXAMPLES

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. Definitions of variables in the structures in schemes herein are commensurate with those of corresponding positions in the formulae delineated herein.

The synthesis of the compounds of the invention is provided in PCT/US2011/060791, which is incorporated herein by reference in its entirety.

### Example 1

### Bone Marrow Differentiation of B Cells is Altered in Diseased NZB/W Mice

B cells originate from pluripotent hematopoietic stem cells in the bone marrow. Once the B cell pathway has been selected, B cell development and differentiation occurs in a series of stages, progressing from pro- to pre-, to immature B cells (Hardy et al Annual review of immunology 2001, 19:595-621). Pro-B cells (B220+CD43+) pass through 4 developmental phases: A (CD24−BP1−), B (CD24+BP1−), C (CD24loBP1+), and C′ (CD24hiBP1+), while undergoing heavy chain D-J and V(D)J rearrangement (Hardy et al. J. Exp. Med. 1991, 173(5):1213-1225; Alt et al. EMBO J. 1984, 3(6):1209-1219). Following successful IgG heavy chain rearrangement, CD43 expression is downregulated and cells progress into the pre-B cell (B220+CD43−) phase. Pre-B cells pass through 3 fractions: D (IgM−IgD−), E (IgM+IgD−), and F (IgM+IgD+) (Hardy et al. J. Exp. Med. 1991, 173(5):1213-1225). Fraction D cells rearrange Ig light chains, begin to express IgM and differentiate into fraction E or immature B cells (Ehlich et al. Cell 1993, 72(5):695-704). Fraction E cells exit the bone marrow and continue to mature in the spleen. As IgM+ immature B cells begin to express IgD, they progress into fraction F, or mature B cells.

Systemic lupus erythematosus (SLE) is an autoimmune disease that can affect nearly every organ in the body. A pathognomonic feature of lupus is B cell dysregulation leading to autoantibody production and immune-complex mediated glomerulonephritis. Hyperactive B cells contribute to SLE pathogenesis by inducing CD4+ T helper cells, inhibiting regulatory T (Treg) cells, secreting proinflammatory cytokines, and producing autoantibodies. Reduced Treg cell numbers and function have been reported during active SLE, which contributes to immune dysregulation and a lack of self-tolerance.

Female New Zealand Black/White (NZB/W) mice mimic human disease in several ways and therefore serve as an acceptable mouse model of SLE. NZB/W mice are generated from the cross of New Zealand Black/BinJ (NZB) and New Zealand White/LacJ (NZW) mice and develop a spontaneous lupus-like disease (Ryan et al. Am J Physiol Regul Integr Comp Physiol 2007, 292(2):R736-742; Burnett et al. Exp Toxicol Pathol 2004, 56(1-2):37-44).Both NZB/W mice and humans with active SLE produce autoantibodies against double-stranded DNA (dsDNA) and histones and develop immune complex-mediated glomerulonephritis (Haas et al. J. Immunol. 2010, 184(9):4789-4800; Ebling et al. 1989, 5(1):79-95; Theofilopoulos et al. Advances in Immunology 1985, 37:269-390).

To determine if B cell development was altered in the bone marrow of lupus prone NZB/W mice, pro- and pre-B cell differentiation was first evaluated in pre-diseased and diseased NZB/W mice.

### Mice

Female NZB/W F1 mice were purchased from Jackson Laboratories (Bar Harbor, Me., USA). All mice were used in accordance with the Institutional Animal Care and Use Committee of Virginia Polytechnic Institute and State University (Virginia Tech) and housed in the animal facility at the Virginia-Maryland Regional College of Veterinary Medicine (VMRCVM, Blacksburg, Va., USA).

Isolation of B Cells from the Bone Marrow

Bone marrow cells were flushed in PBS with 1% BSA from the femurs of NZB/W mice following euthanization. Red blood cells (RBCs) were lysed using ammonium chloride potassium (ACK) lysing solution. Single-cell suspensions were then washed and stained with Allo-Phycocyanin (APC)-conjugated B22 and Flourescein Isothiocyanate (FITC)-conjugated CD43 anti-mouse mAbs to identify pro-B cell (B220⁺ CD43⁺) and pre-B cell (B220⁺ CD43⁻) populations. Pro-B cell populations were further stained with Phycoerythirn (PE)-conjugated BP1 and PECy5 or Peridinin-chlorophyll proteins (PerCP)-conjugated CD24 anti-mouse mAbs to identify fractions A (B220⁺ CD43⁺ CD24⁻ BP1⁻), B (B220⁺ CD43⁺ CD24⁺ BP1⁻), C (B220⁺ CD43⁺ CD24^{lo} BP1), and C′ (B220⁺ CD43⁺ CD24^{hi} BP1⁺). Pre-B cells fractions were further stained with PE-conjugated IgD, and PECy5-conjugated IgM anti-mouse mAbs to identify fractions D (B220⁺ CD43⁻ IgM⁻ IgD⁻), E (B220⁺ CD43⁻ IgM⁺ IgD⁻) and F (B220⁺ CD43⁻ IgM⁺ IgD⁺). Fractions were measured by flow cytometric analysis. All antibodies were purchased from eBioscience (San Diego, Calif., USA). Flow cytometry was performed at that College of Veterinary Medicine Flow Cytometry Core Facility using a FACScan flow cytometer (Becton Dickinson, Franklin Lakes, N.J., USA). Flow cytometry data was analyzed using FlowJo Software (Tree Star, Ashland, Oreg., USA).

### Evaluation of Pro- and Pre-B Cells in NZB/W Mice

Bone marrow cells were harvested from pre-diseased and diseased NZB/W mice and labeled with fluorescently tagged antibodies specific for pro- and pre-B cells.

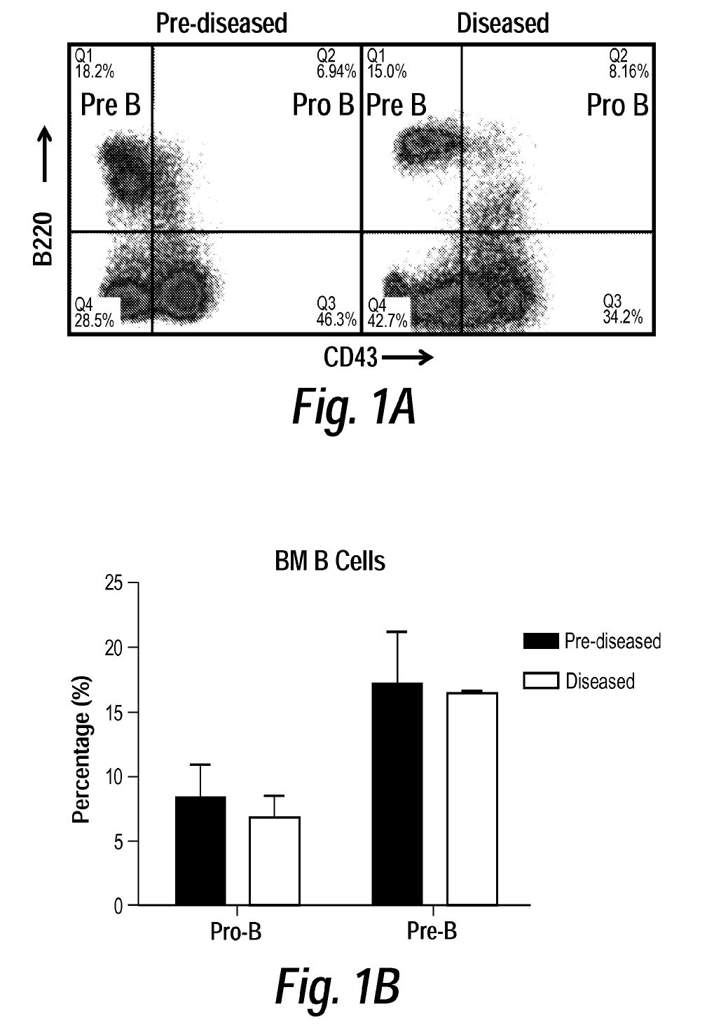

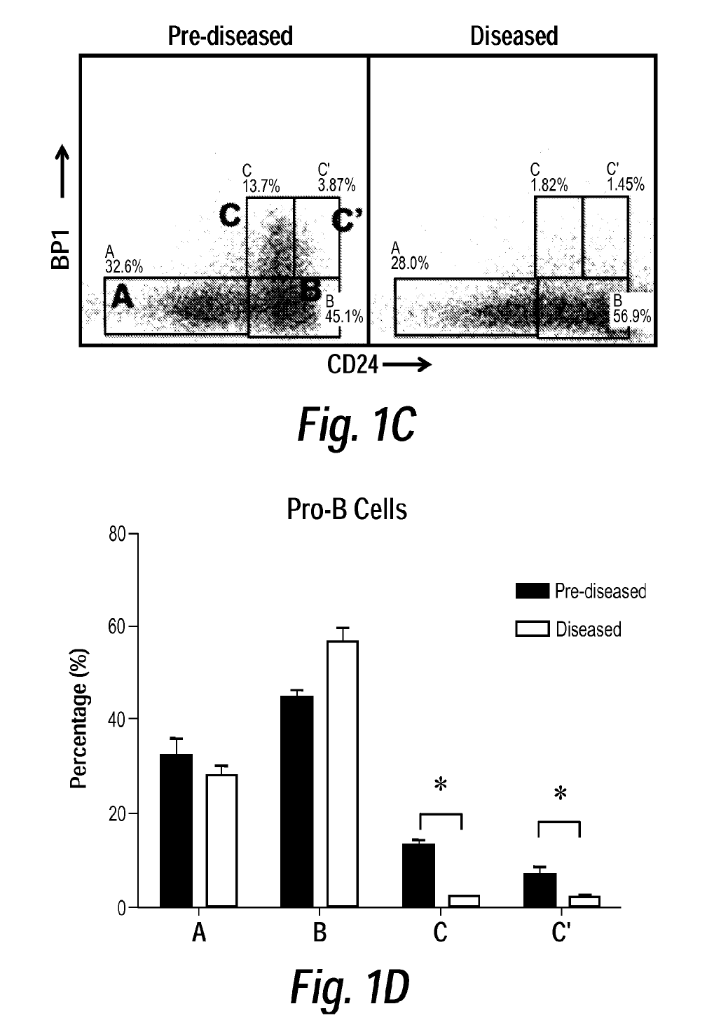

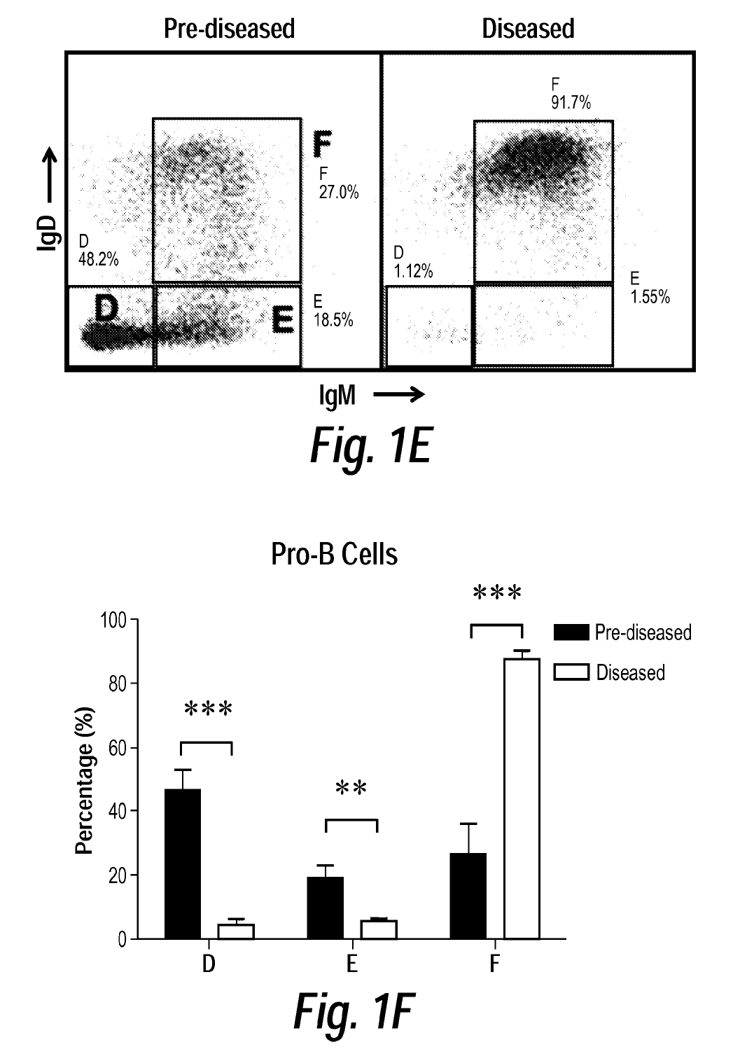

Bone marrow cells were harvested from NZB/W mice at 8 (pre-diseased) or 38 weeks-of-age (diseased) and sorted into pro-B cell (CD43+B220+) and pre-B cell (CD43−B220+) populations (FIG. 1 A-B). FIG. 1A shows representative images of B cells labeled with CD43 and B220. FIG. 1B shows that there were no significant differences in the percentages of pro- or pre-B cells between diseased and pre-diseased NZB/W mice (FIG. 1 B).

To characterize B cell development further, pro-B cells were divided into developmental fractions A (B220+CD43+CD24−BP1−), B (B220+CD43+CD24+BP1−), C (B220+CD43+CD24lo BP1+), and C′ (B220+CD43+CD24hi BP1+) (FIGS. 1 C-D). FIG. 1C shows a representative flow cytometry image of pro-B cell fractions A, B, C, and C′ from pre-diseased and diseased NZB/W mice. Diseased NZB/W mice had significantly fewer cells in fractions C and C′ when compared to pre-diseased mice (FIG. 1 D).

Pre-B cells were further divided into fractions D (B220+CD43−IgM−IgD−), E (B220+CD43−IgM+IgD−) and F (B220+CD43−IgM+IgD+) (FIGS. 1 E-F). Diseased NZB/W mice had markedly fewer cells in fractions D and E, but a significant increase in the percentage of cells in fraction F (see FIG. 1F).

### Conclusion

Diseased NZB/W mice have increased survival of cells during bone marrow B cell differentiation leading to an accumulation of cells in the late pre-B cell fraction F. These results suggest that bone marrow B cell development progresses more rapidly through developmental stages during SLE disease, without the proper removal of defective B cells leading to an increased number of auto-reactive B cells leaving the bone marrow.

### Example 2

### Treatment of Diseased NZB/W Mice with ACY-738 Alters Bone Marrow B Cell Differentiation In Vivo

NZB/W mice were treated with an HDAC6 inhibitor to determine if it altered the B cell differentiation profile observed in Example 1.

### In Vivo Treatment

NZB/W mice were injected intraperitoneally 5 days/week with the vehicle control (DMSO), ACY-738 treatment at 5 mg/kg (low-dose), or ACY-738 treatment at 20 mg/kg (high-dose) beginning at 22-weeks-of-age until euthanization at 38 weeks-of-age. ACY-738 was received from Acetylon Pharmaceuticals for use in all studies. Proteinuria and weight were measured every 2 weeks and blood was collected every four weeks for sera analysis. Proteinuria was measured by a standard semi-quantitative test using Siemens Uristix dipsticks (Siemens Healthcare, Deerfield, Ill., USA). Results were quantified according to the manufacturer's instructions and scored as follows: dipstick reading of 0 mg/dL=0, trace=1, 30-100 mg/dL=2, 100-300 mg/dL=3, 300-2000 mg/dL=4, and 2000+ mg/dL=5.

### HDAC6 Inhibition Altered Pro- and Pre-B Cell Populations in the Bone Marrow.

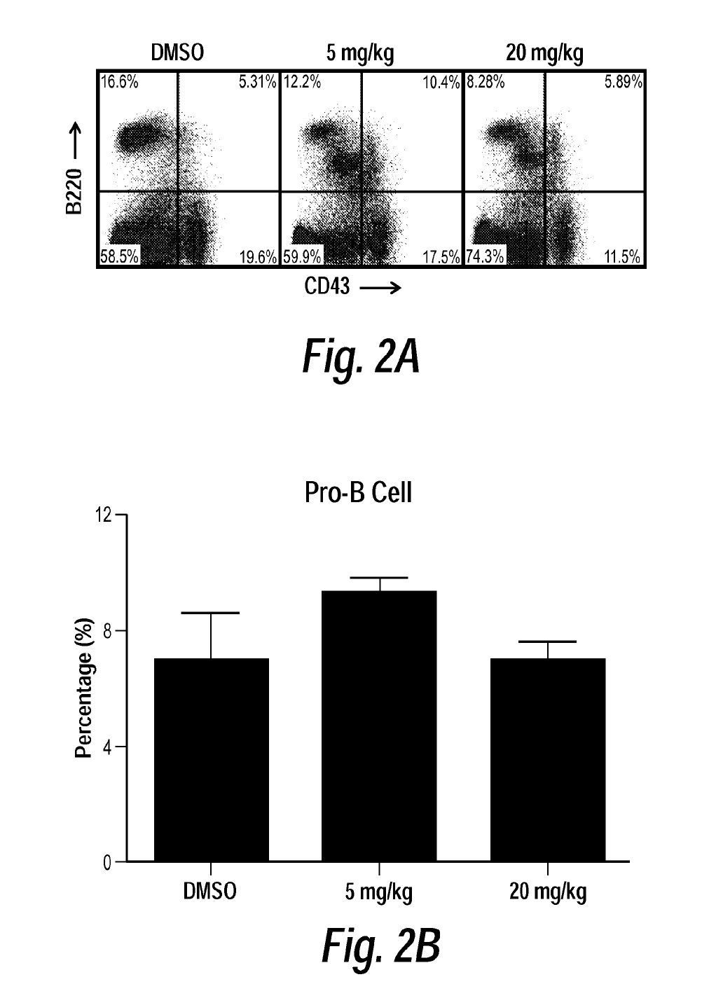

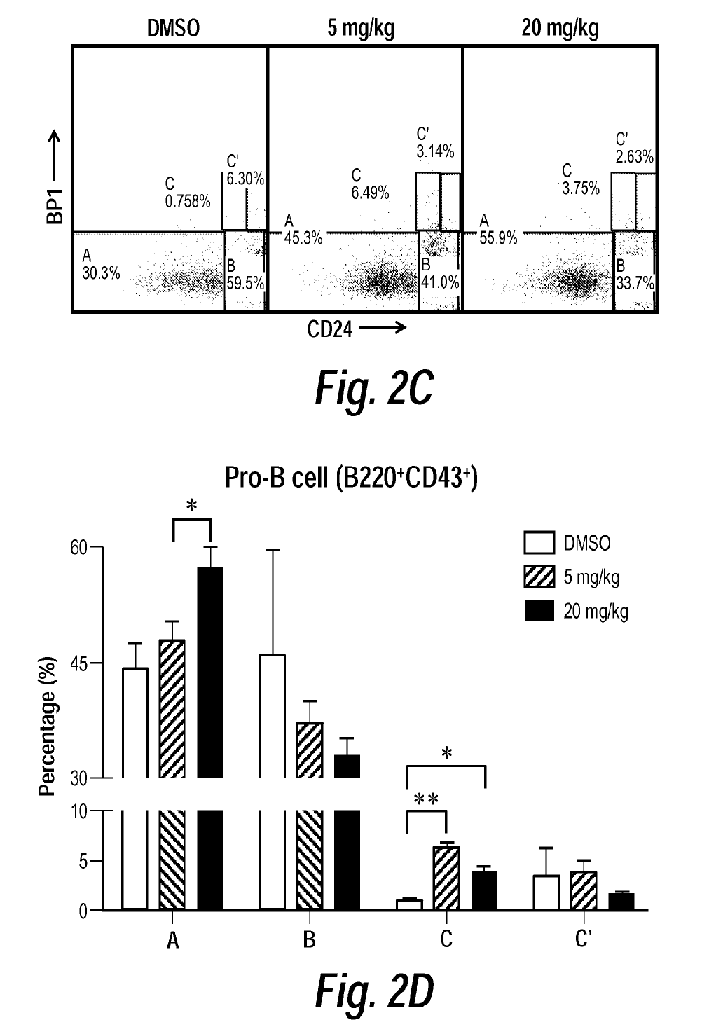

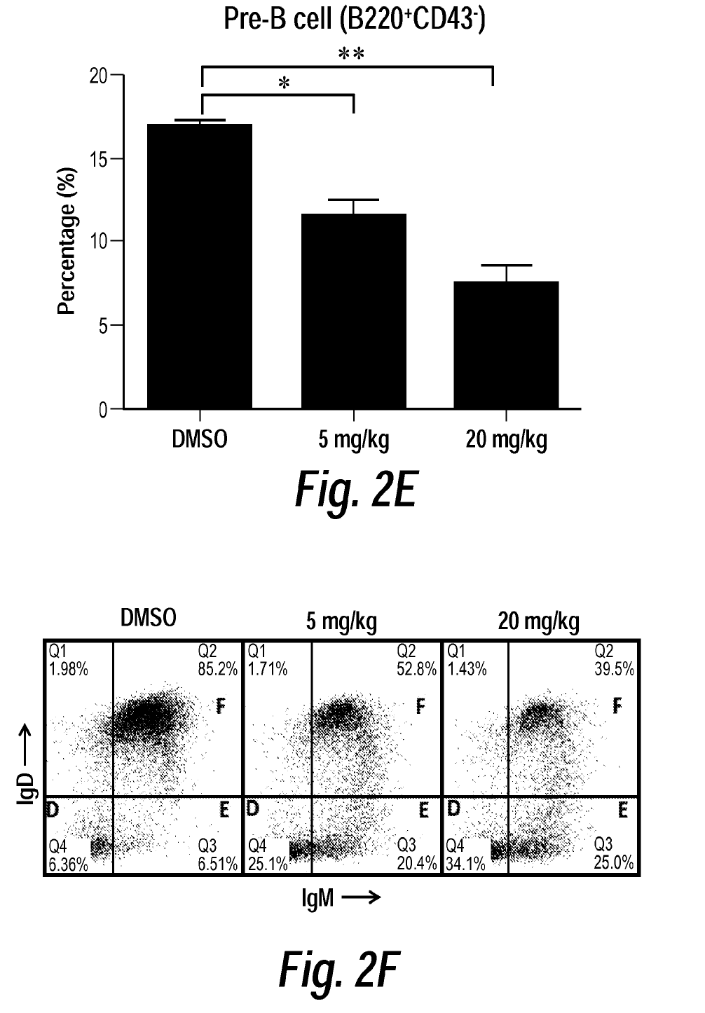

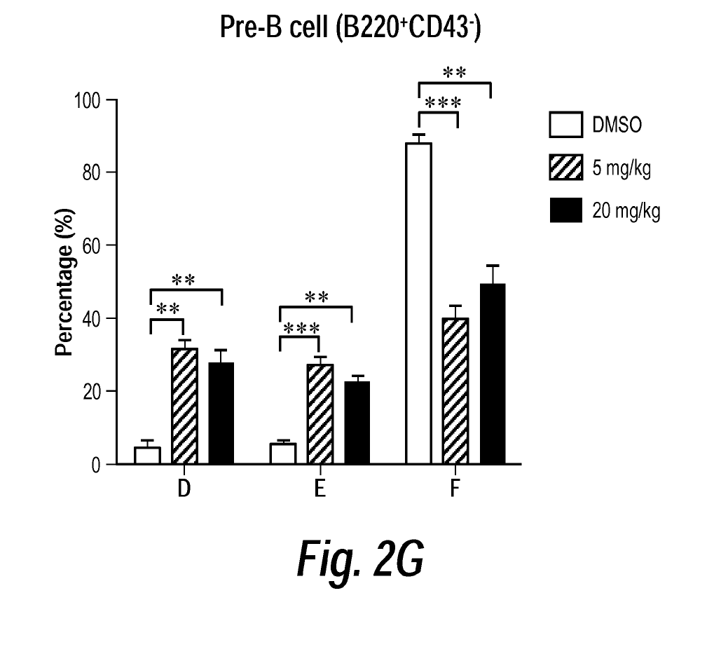

Following treatment with ACY-738, harvested bone marrow cells were stained with B220 and CD43 and the percentage of pro- and pre-B cells was determined using flow cytometric analysis. FIG. 2A depicts a representative flow cytometry image of pro-B cell (B220⁺ CD43⁺) and pre-B cell (B220⁺ CD43⁻) populations. FIG. 2B shows that the percentage of pro-B cells was not significantly altered following HDAC6 inhibition.

FIG. 2C shows a representative flow diagram of pro-B cell fractions: A, B, C, and C′. FIG. 2D shows that treatment with ACY-738 significantly increased the percentage of pro-B cells in fractions A and C. In contrast, the pre-B cell population was significantly decreased in a dose-dependent manner following HDAC6 inhibition in NZB/W mice (see FIGS. 2A and 2E).

FIG. 2F shows a representative flow cytometry image of pre-B cell fractions D, E, and F. The developmental pre-B cell stages were also significantly altered. At both the high and low dose of ACY-738, there was a significant increase in the percentage of cells in fractions D and E that corresponded with a decrease in cells in fraction F (see FIG. 2G).

### Conclusion

HDAC6 inhibition is able to reduce the number of cells that survive bone marrow differentiation resulting in fewer B cells continuing development in the periphery.

### Example 3

### Splenic and Peripheral B Cell Populations were not Significantly Altered by HDAC6 Inhibition

Previous studies have shown abnormal numbers of splenic B cells from SLE patients in the transitional and MZ developmental stages (Wither et al. Clinical immunology 2000, 94(1):51-63; Grimaldi et al. Journal of immunology 2001, 167(4):1886-1890). In addition, major differences in peripheral B cells between SLE patients and healthy controls have also been reported (Korganow et al. Journal of autoimmunity 2010, 34(4):426-434; Odendahl et al. Journal of Immunology 2000, 165(10):5970-5979).

NZB/W mice were therefore treated with ACY-738 to determine if HDAC6 inhibition can correct the abnormal splenic and peripheral B cell populations seen in SLE.

### Isolation of Splenic B Cells

Following euthanization, spleens were removed and single-cell suspensions of splenocytes were incubated with PerCP710 conjugated IgM, FITC conjugated AA4.1, PE conjugated CD23, and APC conjugated CD21 anti-mouse mAbs (eBioscience, San Diego, Calif., USA). IgM⁺ cells were analyzed for the expression of AA4.1, CD23 and CD21 and divided into the following developmental stages using flow cytometry: T1 (IgM⁺ CD23⁻ AA4.1⁺ CD21⁻), T2 (IgM^{hi} CD23⁺ AA4.1⁺ CD21⁺), T3 (IgM^{lo} CD23⁺ AA4.1⁺ CD21⁺), F₀ (IgM⁺ CD23⁺ AA4.1⁻ CD21⁻), MZ (IgM⁺ CD23⁻ AA4.1⁻ CD21⁺) or B1 (IgM⁺ CD23⁻ AA4.1⁻ CD21⁻). Flow cytometry data was analyzed using FlowJo.

### Isolation of Peripheral B Cells

Blood was collected prior to euthanization using retro-orbital bleeding. RBCs from the peripheral blood were lysed and single cell suspensions were labeled using FITC-conjugated B220 and PerCep710 conjugated IgM anti-mouse mAbs (eBioscience, San Diego, Calif., USA). Mature B cells were identified as IgM⁺B220⁺ cells. Flow cytometry data was analyzed using FlowJo.

Splenic and Peripheral B Cell Populations were not Significantly Altered by HDAC6 Inhibition.

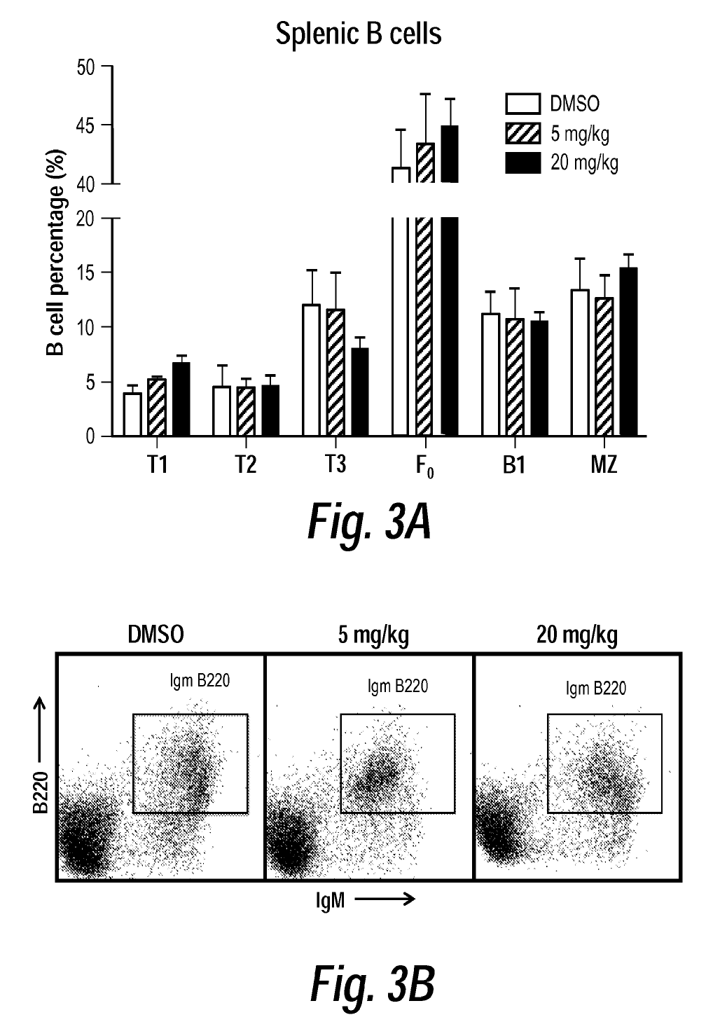

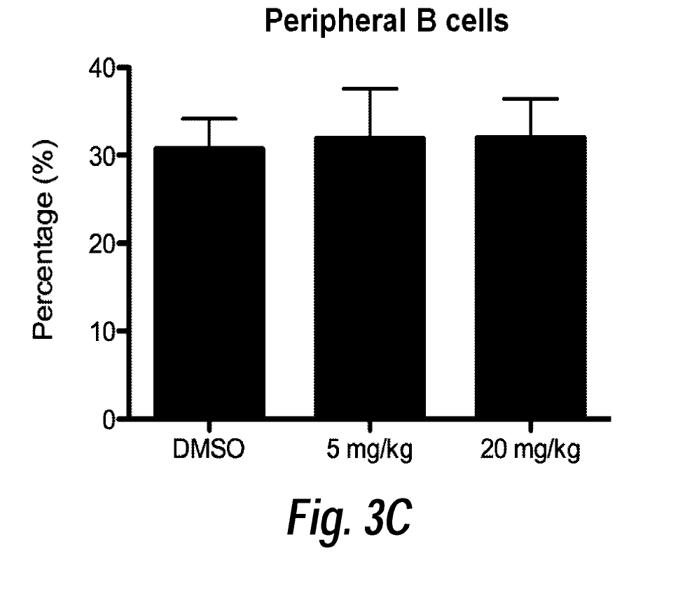

After staining with PerCP710 conjugated IgM, FITC conjugated AA4.1, PE conjugated CD23, and APC conjugated CD21 anti-mouse mAbs, a single cell suspension of splenocytes was divided into developmental stages T1 (IgM⁺ CD23⁻AA4.1⁺ CD21⁻), T2 (IgM^{hi} CD23⁺ AA4.1⁺ CD21⁺), T3 (IgM^{lo} CD23⁺ AA4.1⁻CD21⁺), F₀ (IgM⁺ CD23⁺ AA4.1⁻CD21⁻), MZ (IgM⁺ CD23⁻AA4.1⁻CD21⁺), and B1 (IgM⁺ CD23⁻AA4.1⁻CD21⁻). Treatment with ACY-738 did not significantly affect these populations of B cells at either the low or high dose (FIG. 3A).

Similarly, a single-cell suspension of peripheral B cells was obtained from the blood of 38-week-old NZB/W mice and stained for IgM and B220. FIG. 3B shows a representative flow cytometry diagram of peripheral B cells (IgM⁺ B220⁺). Treatment with ACY-738 had no effect on the percentage of peripheral B cells (IgM+B220+) in 38-week-old NZB/W mice at either the low or high dose HDAC6i (FIG. 3C).

### Conclusion

Splenic and peripheral B cell populations were not significantly altered by HDAC6 inhibition.

### Example 4

### Inhibition of HDAC6 Alters Thymic T Cell Development

SLE patients and lupus-prone murine models have been reported to have abnormal expression of T cells (Tsokos et al. Clinical Immunology and Immunopathology 1983, 26(2):267-276; Crispin et al. Journal of Immunology 2008, 181(12):8761-8766; Hammond et al. The Journal of Experimental Medicine 1993, 178(6):2225-2230).

NZB/W mice were therefore treated with ACY-738 to determine if HDAC6 inhibition can affect T lymphocyte development by altering the percentage of DN (CD3+CD4−CD8−) T cells.

### Isolation of T Cells

A single-cell suspension was obtained from the thymuses and spleens of treated NZB/W mice at 38 weeks-of-age. Briefly, the thymus was removed from each NZB/W mouse and dissociated across a sterile wire mesh in a petri dish containing ice-cold RPMI 1640 medium (Thermo Scientific). RBCs were lysed using RBC lysis buffer and cells were pelleted and washed with PBS. Splenocytes were stained with APC-conjugated CD3, FITC-conjugated CD4, eFluor450 (eF450)-conjugated CD8a, PerCP-CY5.5-conjugated CD25, and PE-conjugated Foxp3. Thymocytes were stained with APC-CD3, FITC-CD4, and PE-CD8 anti-mouse mAbs (eBioscience, San Diego, Calif., USA). Fluorescence was measured using a FACScan flow cytometer and data was analyzed by FlowJo software.

### The Percentage of DN Thymic T Cells is Reduced Following HDAC6 Inhibition.

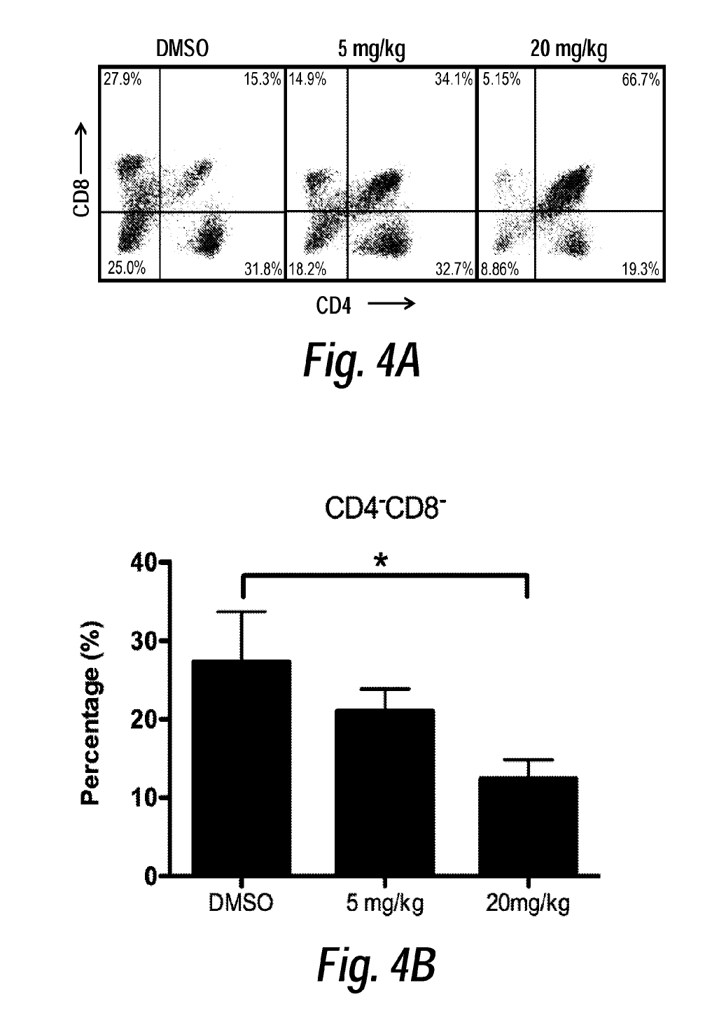

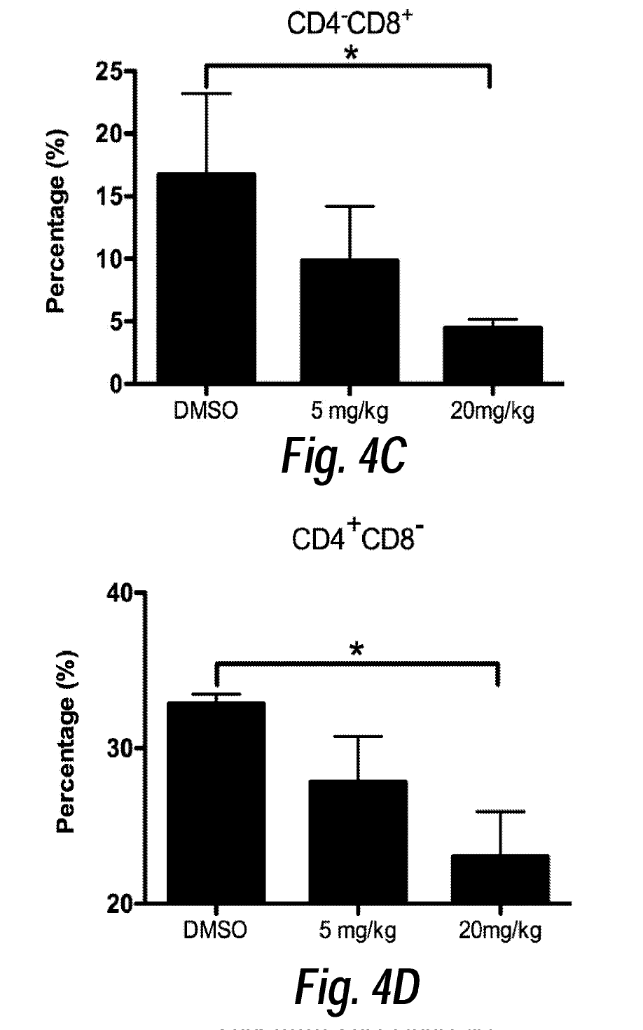

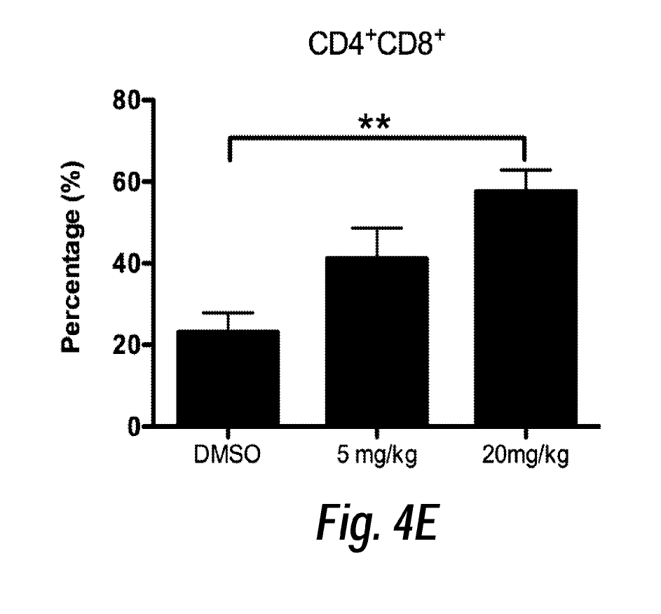

FIG. 4A depicts representative flow cytometry images of thymocytes gated on CD3 and labeled with CD4 and CD8. FIG. 4B shows that ACY-738 treatment decreased the percentage of DN T cells in a dose-dependent manner. HDAC6 inhibition also resulted in a substantial decrease in the percentage of CD3+CD4−CD8+ (FIG. 4C) and CD3+CD4+CD8− single positive (SP) T cells (FIG. 4D). Double positive (CD3+CD4+CD8+) thymic T cell numbers were increased in a dose-dependent manner following 16 weeks of treatment with ACY-738 (FIG. 4E).

### Conclusion

HDAC6 inhibition resulted in a substantial decrease in double negative (DN) CD4⁻ CD8⁻ T cells coupled with a significant increase in double positive (DP) (CD3⁺ CD4⁺ CD8⁺) T cells.

### Example 5

### Inhibition of HDAC6 Increased the Number of Regulatory T Cells in the Spleen

NZB/W mice were treated with ACY-738 to determine if HDAC6 inhibition alters the overall number and function of T_{reg} cells in lupus prone NZB/W mice.

### Specific HDAC6 Inhibition Increased the Treg Phenotype in NZB/W Mice

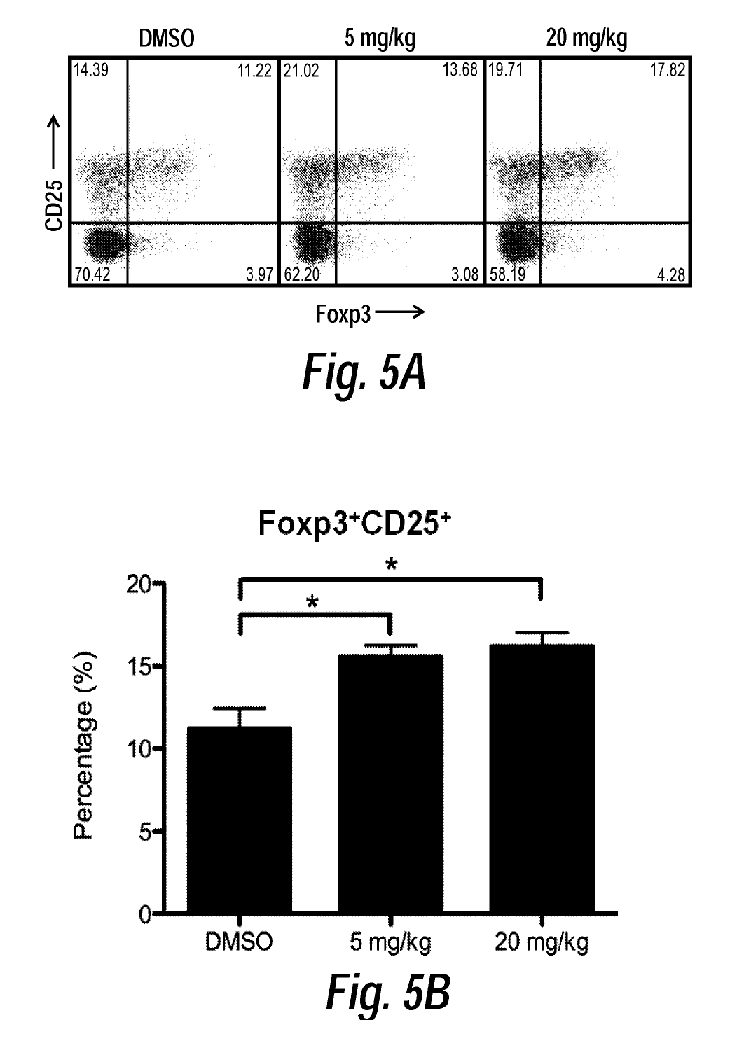

Splenocytes were obtained from 38-week-old NZB/W mice and stained with CD4, CD25, and Foxp3. FIG. 5A shows a representative flow diagram of splenocytes gated on CD4 and labeled with Foxp3 and CD25. FIG. 5B shows that treatment with ACY-738 significantly increased the percentage of T_{reg} cells (CD4⁺ Foxp3⁺ CD25⁺) at both doses compared to mice treated with vehicle control alone (FIG. 5 A-B).

### Example 6

### HDAC6 Inhibition Prolonged Survival of NZB/W Mice and Decreased Urinary Markers of SLE and Splenomegaly

HDAC6 inhibition was evaluated for its efficacy in decreasing SLE markers of disease and prolonging the survival of NZB/W mice.

### Assessment of Survival Rate and Disease Progression in NZB/W Mice

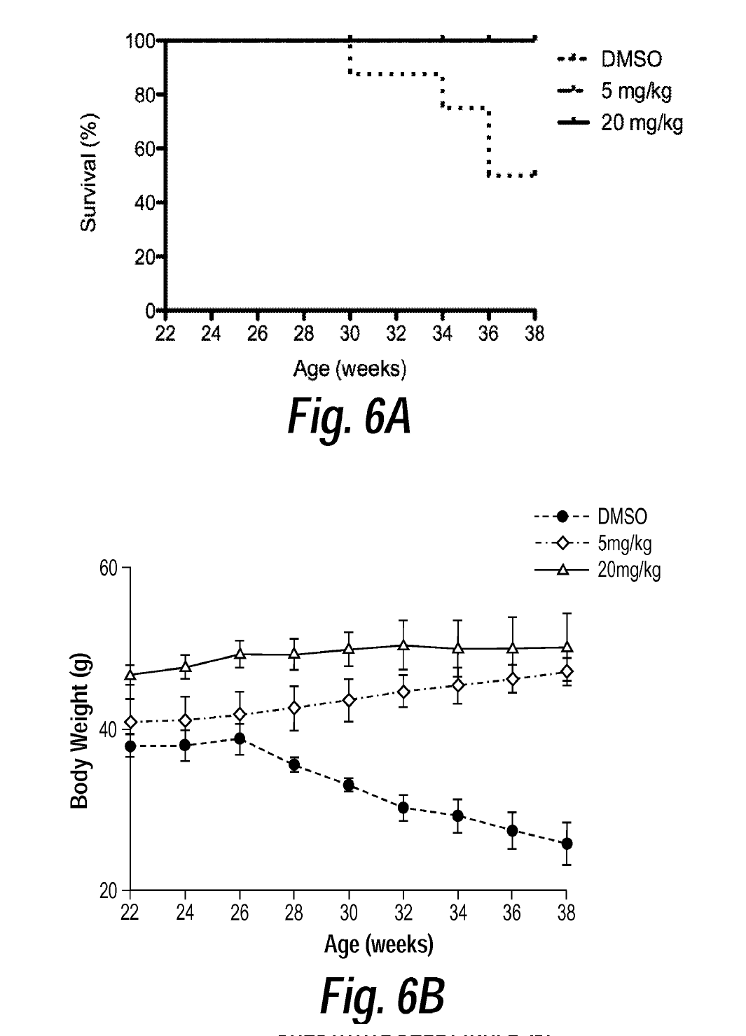

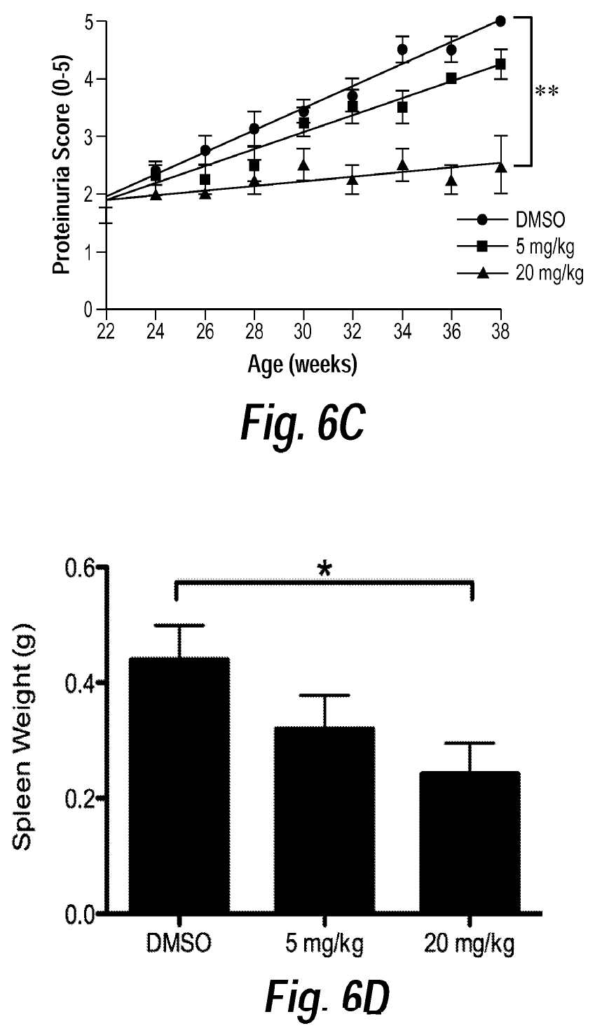

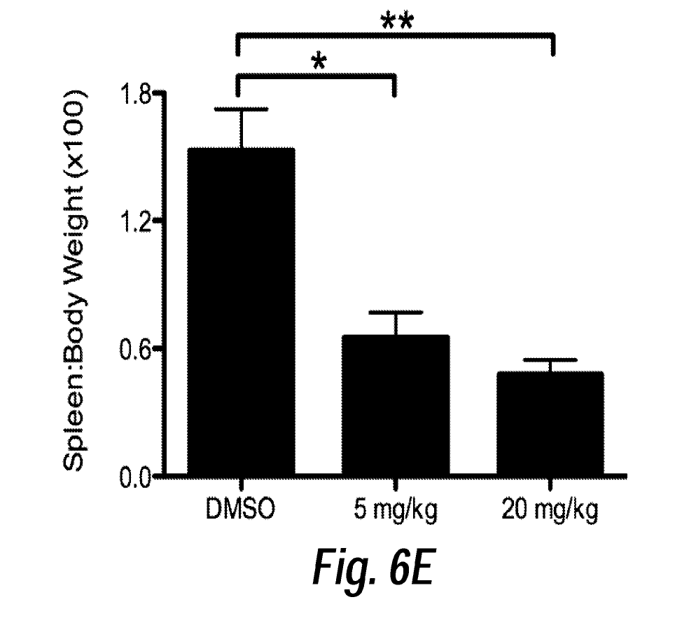

All mice receiving either the high or low dose of ACY-738 survived to the completion of the study. However, half of the NZB/W mice receiving the vehicle control alone died before termination of the study at 38 weeks-of-age (FIG. 6 A). Body weight increased as NZB/W mice aged following treatment with ACY-738. Vehicle control-treated NZB/W mice experienced weight loss concomitantly with increased proteinuria (FIG. 6 B).

Throughout the treatment period, NZB/W mice were monitored for changes in proteinuria and body weight. FIG. 6B shows the measurement of proteinuria every 2 weeks in NZB/W mice being treated with ACY-738 (5 mg/kg in DMSO), ACY-738 (20 mg/kg in DMSO) or vehicle control (DMSO) from 22-38 weeks-of-age. Proteinuria gradually increased as the NZB/W mice treated with the vehicle control or the low-dose of ACY-738 aged. However, treatment with the high-dose of ACY-738 prevented proteinuria from increasing in NZB/W mice. Treatment with 20 mg/kg ACY-738 significantly attenuated the severity of proteinuria in NZB/W F1 mice (FIG. 6 C).

Because SLE is characterized by enlargement of the spleen, splenomegaly was assessed in mice following euthanization by determining the total spleen weight along with the spleen: body weight ratio. Both spleen weight and spleen: body weight ratio were decreased following HDAC6 inhibition in a concentration dependent manner (FIG. 6 D-E).

### Example 7

### Treatment with ACY-738 Reduced Serum Anti-dsDNA and Altered Ig Isotype Levels

NZB/W mice were treated with ACY-738 to determine if HDAC6 inhibition reduces Ig isotype levels and serum anti-dsDNA Ig isotype levels.

### Measurement of Autoantibodies

Sera were collected prior to initiation of treatment at 22 weeks-of-age and every 4 weeks until euthanization. The mice were anesthetized using isoflurane (Piramal Healthcare, Mumbai, Maharashtra, India) and bled from the retro-orbital sinus. Blood was allowed to clot for 2 hours and then centrifuged for 15 min at 10,000×g. The levels of sera antibodies to dsDNA were measured by ELISA. Sera samples were added to the plate at a 1:100 dilution, followed by a two-fold serial dilution. The plate was read at 380 nm on a Spectramax 340PC microplate spectrophotometer (Molecular Devices, Sunnyvale, Calif., USA). A final dilution of 1:800 was reported.

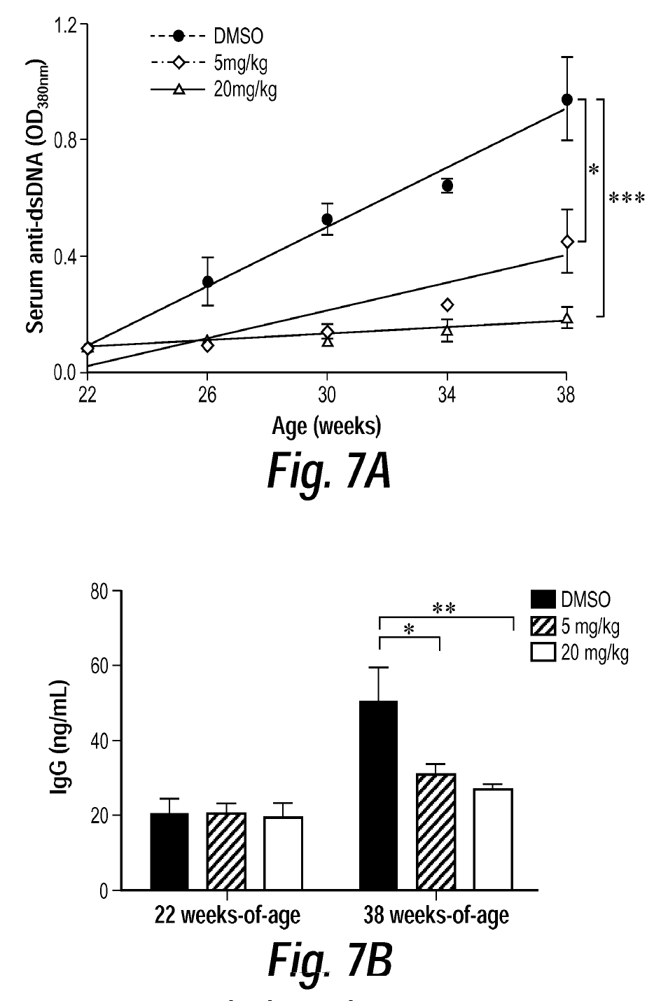

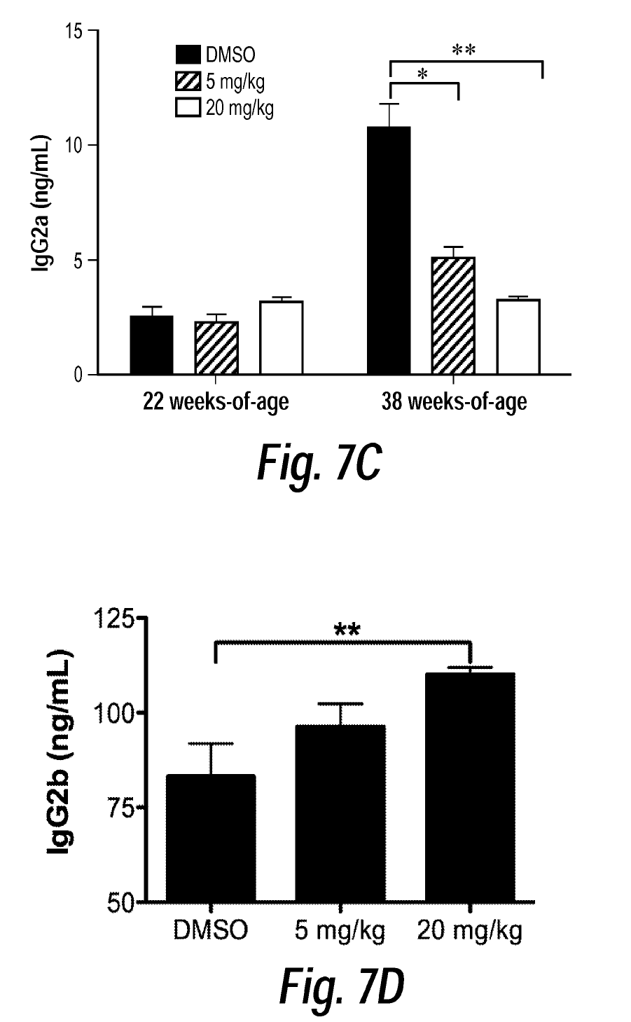

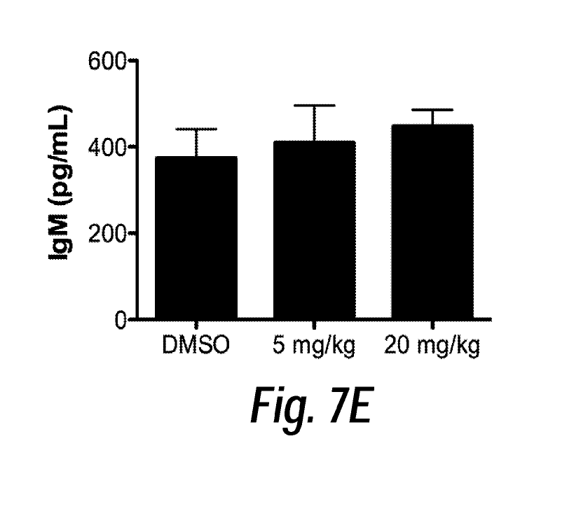

Anti-dsDNA, IgG2a, and total IgG levels gradually increased as the mice aged (FIG. 7 A-C). FIG. 7 A depicts the measurement of sera anti-dsDNA in NZB/W mice at 22 weeks-of-age (prior to treatment) and 38 weeks-of-age (following 16 weeks of treatment). There were no significant differences in anti-dsDNA levels prior to the initiation of treatment. Anti-dsDNA increased in the NZB/W mice as they aged; however, treatment with the high dose of ACY-738 was able to prevent an increase in anti-dsDNA as the mice aged. Even NZB/W mice that received 5 mg/kg ACY-738 showed a significant decrease in anti-dsDNA production as they aged when compared to the vehicle control-treated mice (p<0.05). Following treatment with 20 mg/kg dose, the decrease in autoantibody production was more pronounced compared to NZB/W mice that received the lower dose or vehicle control alone (FIG. 7 A).

FIG. 7B shows that there were no significant differences in sera IgM levels at 22 weeks, but ACY-738 treatment significantly decreased the level of IgG in a dose-dependent manner.

FIG. 7C shows that anti-dsDNA levels increased over time, but ACY-738 treatment significantly decreased the level of IgG2a in a dose-dependent manner.

FIG. 7D shows that, at 38 weeks-of age, treatment with the ACY-738 significantly increased IgG2b.

FIG. 7E shows that at 38 weeks-of-age, total IgM was slightly increased in mice that received ACY-738.

### Conclusion

The treatment of NZB/W mice with ACY-738 significantly decreased levels of IgG2a and total IgG, but increased levels of IgG2b in a dose-dependent manner (FIG. 7 B-D). HDAC6 inhibition had no effect on levels of IgM in mice at 38 weeks-of-age (FIG. 7 E).

### Example 8

### HDAC6 Inhibition Prevented TGF-β AND IL-1β Production from being Altered as NZB/W Mice Aged

NZB/W mice were treated with ACY-738 to determine if HDAC6 inhibition attenuated changes in TGF-β and IL-1β levels seen with the onset of lupus like symptoms.

### ELISA

IL-1β and TGF-β levels were measured from the sera by ELISA according to the manufacturer's protocol (eBioscience, San Diego, Calif., USA). The plate was read at 450 nm on a microplate spectrophotometer.

### Cytokine Production in Lupus-Prone Mice

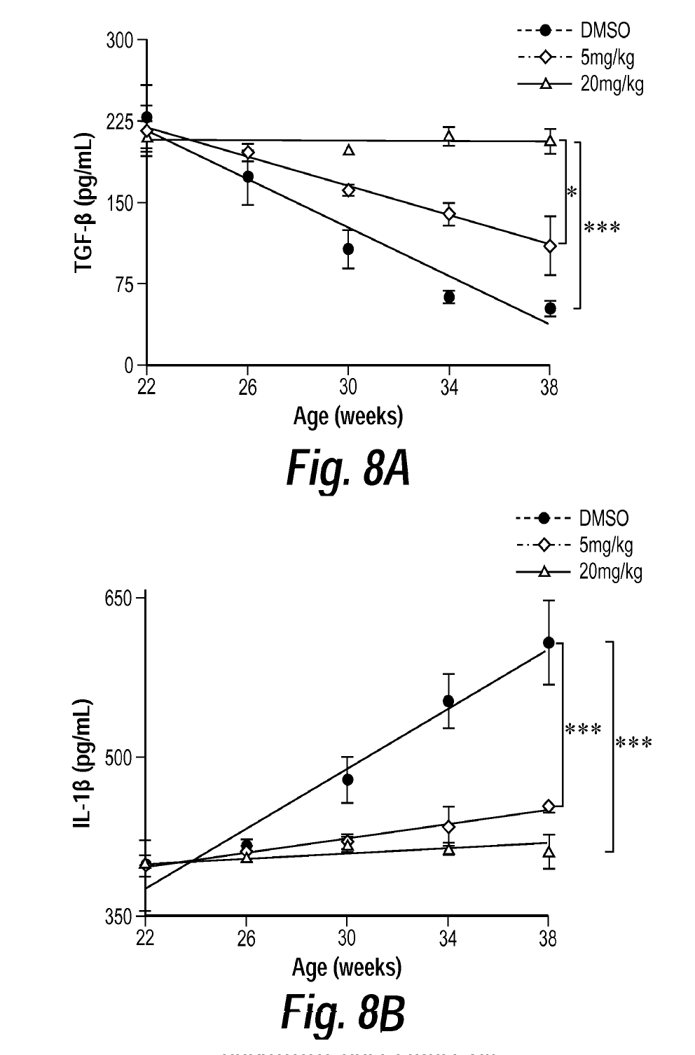

IL-1β and TGF-β levels in the sera of NZB/W mice were measured every 4 weeks starting from 22 weeks until euthanization at 38 weeks-of-age (FIG. 8 A-B). At 22 weeks-of-age there were no significant differences in cytokine levels amongst the three groups. As the NZB/W mice aged, sera levels of TGF-β decreased whereas levels of IL-1β increased (FIG. 8 A-B).

FIG. 8A shows that TGF-β levels significantly decreased in vehicle control-treated mice but that treatment with ACY-738 was able to reverse this trend in a dose-dependent manner. Levels of IL-1β were elevated in 38-week-old NZB/W mice but treatment with ACY-738 significantly decreased sera IL-1β.

### Conclusion

HDAC6 inhibition with ACY-738 attenuated the reduction of TGF-β production in a dose-dependent manner as the mice aged. Treatment with both the low and high dose of ACY-738 also attenuated the increase in sera IL-1β production.

### Example 9

### Glomerular IL-10, TGF-β, and IL-6 mRNA Expression is Decreased Following HDAC Inhibition In Vivo

NZB/W mice develop renal disease around 20 weeks-of-age, progressing to severe glomerulonephritis by 36 weeks-of-age (Dixon FJ Hospital practice 1982, 17(3):63-73). Altered mRNA glomerular expression can lead to fibrosis and irreversible glomerular damage (McMurray et al. Clinical immunology and immunopathology 1997, 84(3):260-268). The balance between Th1 and Th2 cytokines plays a critical role in the immune response and the pathogenesis of autoimmune disease. IL-10 has been reported to be elevated in SLE and plays a critical role in B cell survival, differentiation, and Ig secretion. TGF-β has been shown to play a dual role in SLE pathogenesis. In NZB/W mice, TGF-β is produced in the kidneys to counter inflammation resulting from autoantibody production (Saxena et al. Journal of immunology 2008, 180(3):1903-1912). IL-6 induces polyclonal B-cell activation and autoantibody production during SLE. Studies have also demonstrated increased expression of the proinflammatory cytokine, IL-6, in lupus kidneys (Aringer et al. Lupus 2005, 14(1):13-18).

To determine if HDAC6 inhibition could alter glomerular mRNA expression, ACY-738 was administered to NZB/W mice and glomerular IL-10, TGF-β, AND IL-6 mRNA expression was determined.

### Isolation of RNA

RNA was isolated using the mirVana miRNA isolation kit according to the manufacturer's protocol (Applied Biosystems, Carlsbad, Calif., USA). The eluates were quantified on a spectrophotometer (Nanodrop, Thermo Scientific, Waltham, Mass., USA). An aliquot was taken and diluted to 1 ng/μL for real-time RT-PCR. The eluted RNA was stored at −80° C.

### Real-Time RT-PCR

IL-6, IL-10 and TGF-β mRNA expression were measured using TaqMan Gene Expression assays (Applied Biosystems, Carlsbad, Calif., USA). The ΔΔC_{T} was calculated using the endogenous control GAPDH, and then the ΔC_{T} was determined by calculating the fold change in expression between the NZB/W mice treated with ACY-738 and the DMSO-treated controls. All samples were run in triplicate.

### Glomerular Isolation

The cortical tissue was isolated from one kidney of each mouse and pooled by treatment group. Briefly, cortical tissue was pressed through grading sieves and resuspended in 750 U/mL Worthington type I collagenase at 37° C. for 20 min. Glomerular cells were pelleted, resuspended in RNAlater (QIAGEN, Valencia, Calif., USA), and stored at −20° C. until RNA isolation.

Glomerular mRNA Levels in 38-Week-Old NZB/W Mice

Following euthanization, glomeruli were isolated from the kidneys and RNA was extracted. Relative glomerular mRNA expression of IL-10, TGF-β, and IL-6 were determined using real-time RT-PCR.

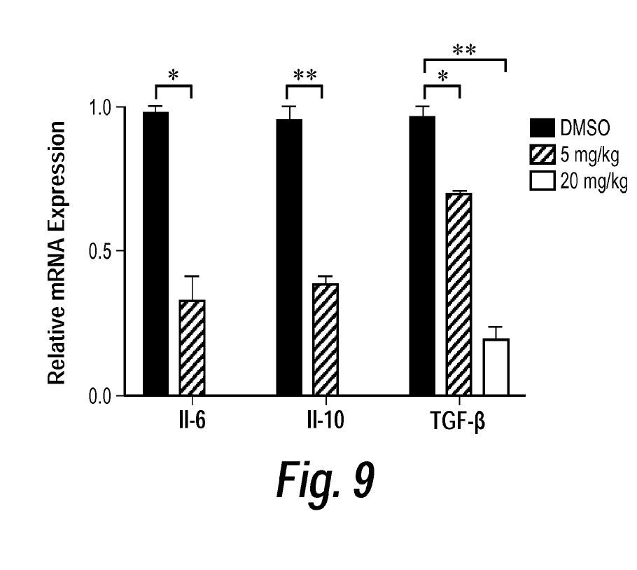

Administration of ACY-738 (5 mg/kg) was able to significantly reduce glomerular IL-6 and IL-10 mRNA levels by more than 50% while treatment with 20 mg/kg ACY-738 reduced IL-6 and IL-10 mRNA to non-detectable levels (FIG. 9 A). Furthermore, glomerular TGF-β mRNA was decreased following inhibition of ACY-738 in a dose-dependent manner (FIG. 9 A).

### Conclusion

Taken together these results suggest that HDAC6 inhibition by ACY-738 reduces the production of SLE-associated cytokine mRNA.

### Example 10

### Glomerular Immune Complex Deposition is Reduced Following HDAC6 Inhibition

NZB/W mice were treated with ACY-738 to determine if HDAC 6 inhibition decreased immune complex deposition and complement activation in renal tissue.

### Pathology

At the time of euthanization, the kidneys were removed and cut in half. One half of the kidney from each mouse was fixed in formalin, embedded in paraffin, sectioned, and stained with Periodic acid-Schiff (PAS). Kidney sections were scored (0-4) for glomerular proliferation, inflammation, crescent formation, necrosis, and by a pathologist, Dr. David Caudell, in a blinded manner.

### Immunofluorescence Staining

One half of each kidney was placed in OCT media and snap-frozen in a slurry containing dry ice and 2-methylbutane (Fisher Scientific, Hampton, N.H., USA). Frozen kidney sections were cut into 3 μM sections and stained with goat anti-mouse IgG conjugated to FITC (Pierce) diluted 1:100 or goat anti-mouse C3-FITC (Pierce, Thermo Fisher Scientific, Waltham, Mass., USA) diluted 1:100. Kidney sections were examined by fluorescent microscopy. Sections were scored (0-4) for immune complex deposition by a pathologist in a blinded manner.

### HDAC6 Inhibition Decreased Glomerular Immune Complex Deposition

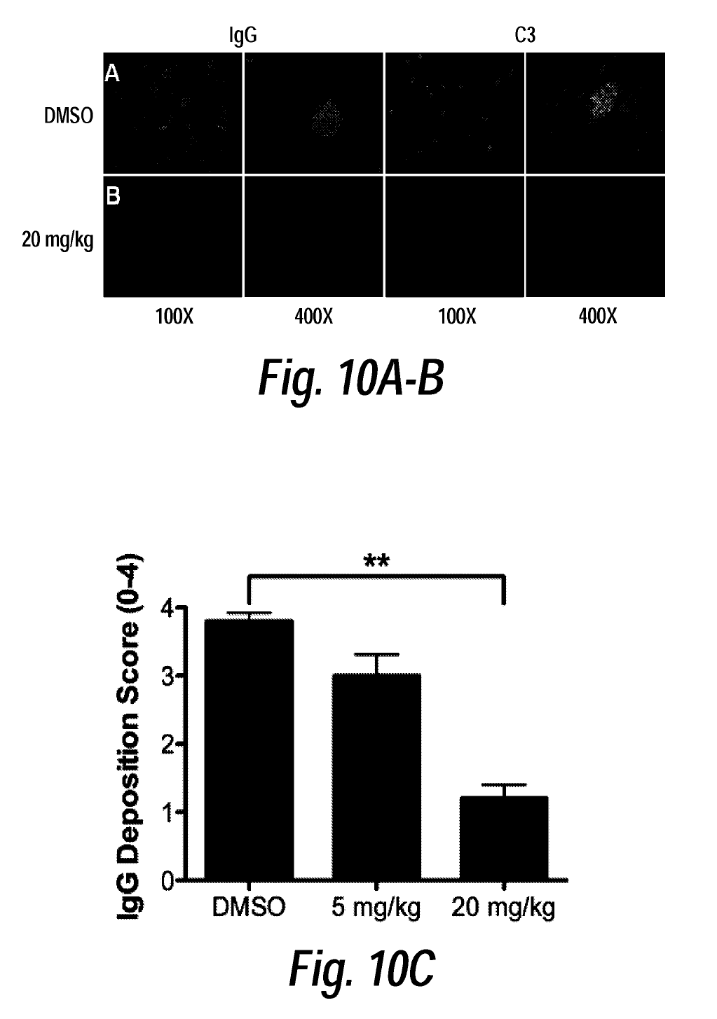

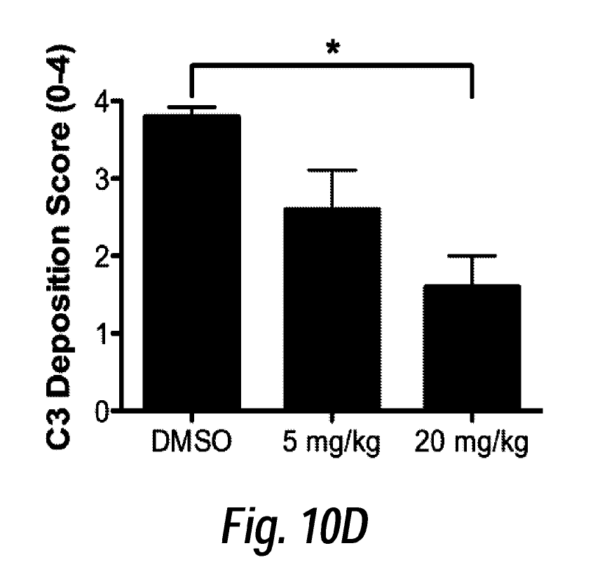

5 μM kidney sections were stained with FITC-conjugated C3 or IgG and assessed for fluorescence intensity. (see FIGS. 10A and 10B). Both IgG and C3 deposition levels were significantly decreased following 16 weeks of ACY-738 treatment.

Treatment with the high-dose of ACY-738 was able to decrease both the number of glomeruli with C3 and IgG deposition as well as the overall level of deposition within each glomerulus (FIG. 10 A). Mice that received 5 mg/kg ACY-738 had a slight decrease in C3 and IgG deposition (data not shown).

Treatment with 20 mg/kg of ACY-738 resulted in decreased IgG and C3 staining compared to vehicle control-treated mice (FIG. 10 B). Each kidney was scored in a blinded manner for fluorescence intensity.

Representative images of glomerular deposition of both C3 and IgG in NZB/W mice treated with the vehicle control or the high dose of ACY-738. can be seen in FIGS. 10C and 10 D. IgG and C3 deposition were evaluated by a pathologist in a blinded manner and scored (0-4) for the level and frequency of fluorescence. Treatment with the 20 mg/kg dose significantly decreased IgG and C3 deposition, however, 5 mg/kg ACY-738 showed no significant effect on IgG and C3 deposition compared to mice that received vehicle control alone.

### Example 11

### SLE-Associated Renal Pathology was Decreased Following ACY-738 Therapy

NZB/W mice were treated with ACY-738 to determine if HDAC 6 inhibition reversed SLE associated renal pathology.

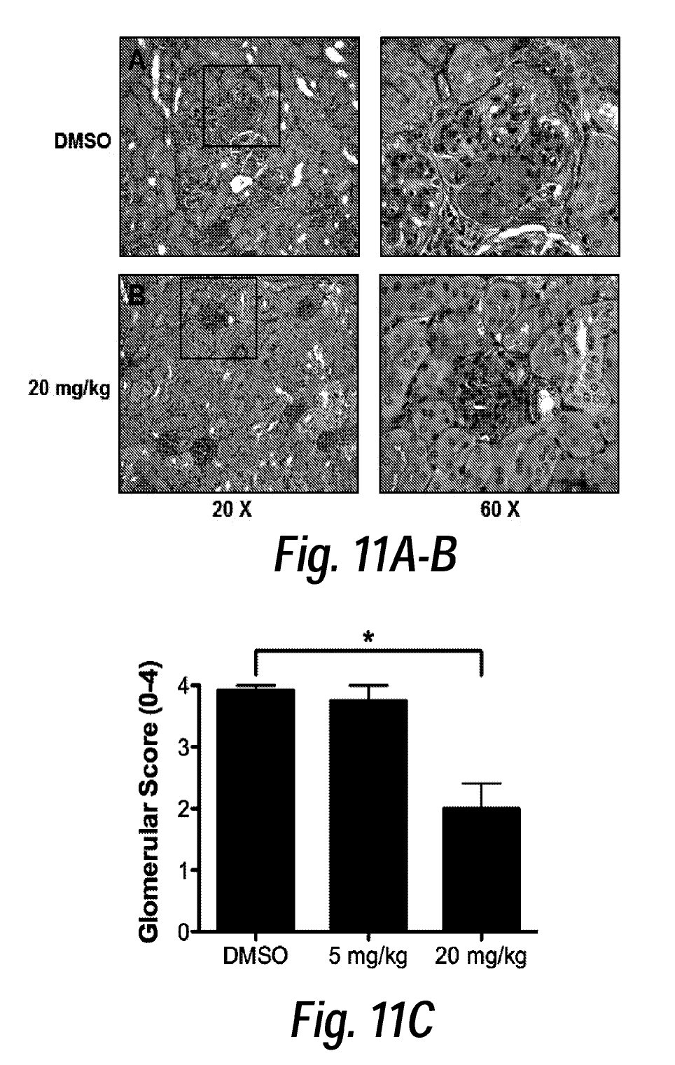

In order to assess renal disease, kidney sections were embedded in paraffin and stained by PAS. NZB/W mice have been shown to develop severe renal disease by 32 weeks-of-age. NZB/W mice treated with DMSO alone had thickened, irregular glomerular basement membranes, increased cellularity, fibrosis and crescent formation (see FIG. 11 A). Treatment with 5 mg/kg ACY-738 did not significantly alter kidney pathology. However, kidneys from mice treated with 20 mg/kg of ACY-738 had significantly reduced SLE renal pathology characterized by the lack of fibrosis and crescent bodies (FIG. 11 B). Sections were assessed for glomerular proliferation, inflammation, number of nuclei per glomerulus, crescent formation, and fibrosis by a blinded pathologist, and a glomerular score (0-4) was assigned. NZB/W mice that were treated with the vehicle control alone received an average score of 4 compared to an average score of 2 from mice that received the high-dose of ACY-738 (FIG. 11 C).

All statistical analyses were performed using Student's unpaired t-test (two-tailed). A linear regression analysis was used to determine the relationship between age and cytokine production following treatment with ACY-738. P values less than 0.05 were considered statistically significant.

1. A method for treating an abnormal lymphocyte function comprising:
administering to a subject in need thereof a therapeutically effective amount of a compound of formula I: 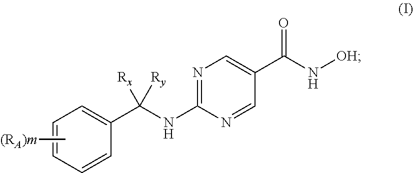
or a pharmaceutically acceptable salt, ester or prodrug thereof,
wherein,
Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;
each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring;
m is 0, 1, or 2; and
wherein the amount of the compound of Formula I is effective at treating the subject's abnormal lymphocyte function.

2. The method for treating an abnormal lymphocyte function according to claim 1, wherein the abnormal lymphocyte function comprises a defect in apoptosis.

3. The method for treating an abnormal lymphocyte function according to claim 2, wherein the defect in apoptosis alters lymphocyte development.

4. The method for treating an abnormal lymphocyte function according to claim 3, wherein the altered lymphocyte development increases the number of immature lymphocytes.

5. The method for treating an abnormal lymphocyte function according to claim 1, wherein the abnormal lymphocyte function produces an auto-reactive lymphocyte.

6. The method for treating an abnormal lymphocyte function according to claim 1, wherein the abnormal lymphocyte function causes an autoimmune disease.

7. The method for treating an abnormal lymphocyte function according to claim 6, wherein the autoimmune disease is a B cell mediated autoimmune disease.

8. The method for treating an abnormal lymphocyte function according to claim 7, wherein the B cell mediated autoimmune disease is systemic lupus erythematosus (SLE).

9. The method for treating an abnormal lymphocyte function according to claim 1, wherein the compound of Formula I is ACY-738.

10. The method for treating an abnormal lymphocyte function according to claim 1, wherein the administration of the compound of Formula I mitigates at least one of the subject's symptoms selected from the group consisting of splenomegaly, aberrant B cell differentiation, an increase in the number double-negative thymic T cells, an increase in the level of anti-dsDNA, immune complex-mediated glomerulonephritis and an increase in inflammatory cytokine production.

11. The method for treating abnormal lymphocyte function according to claim 1, wherein the administration of the compound of Formula I increases the number of the subject's splenic T_{reg} cells.

12. The method for treating abnormal lymphocyte function according to claim 1, wherein the administration of the compound of Formula I increases the percentage of the subject's cells in the early-stage developmental fractions of both pro- and pre-B cells.

13. The method for treating abnormal lymphocyte function according to claim 1, wherein the administration of the compound of Formula I reduces the number of the subject's auto-reactive B cells.

14. A method for reducing the pathogenesis associated with a B cell mediated autoimmune disease comprising:
administering to a subject in need thereof a therapeutically effective amount of a compound of formula I: 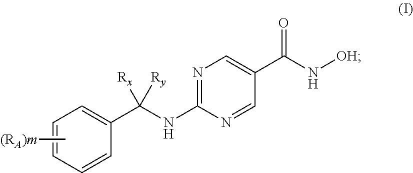
or a pharmaceutically acceptable salt, ester or prodrug thereof,
wherein,
Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;
each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and m is 0, 1, or 2; and
wherein the amount of the compound of formula I is effective at reducing the pathogenesis associated with a B cell mediated autoimmune disease.

15. The method for reducing the pathogenesis associated with a B cell mediated autoimmune disease according to claim 14, wherein the compound of Formula I is ACY-738.

16. The method for reducing the pathogenesis associated with a B cell mediated autoimmune disease according to claim 14, wherein the B cell mediated autoimmune disease is systemic lupus erythematosus.
